(19) United States
(12) Patent Application Publication
(10) Pub. No.: US 2006/0270900 A1
(43) Pub. Date: Nov. 30, 2006

(54) Apparatus and methods for performing ablation

(76) Inventors: Albert K. Chin, Palo Alto, CA (US); Geoffrey H. Willis, Redwood City, CA (US); Shuji Uemura, San Francisco, CA (US); Alfredo R. Cantu, Pleasanton, CA (US); Manuel A. Javier JR., Santa Clara, CA (US); Theodore C. Johnson, Menlo Park, CA (US); Amit Agarwal, Mountain View, CA (US)

Correspondence Address: LAW OFFICE OF ALAN W. CANNON, 834 SOUTH WOLFE ROAD, SUNNYVALE, CA, 94086, US

(21) Appl. No.: 11/138,950

(22) Filed: May 26, 2005

(51) Int. Cl. A61B 1/00; A61B 18/18; A61M 29/00

(52) U.S. Cl. 600/104; 606/1; 606/46; 606/191; 600/176

(57) ABSTRACT

Apparatus and methods for performing endoscopic surgical procedures where only a minimal number of (or even one) openings are required to perform the procedures. Ablation procedures, including epicardial ablation procedures and apparatus for performing such procedures. Epicardial atrial ablation may be performed epicardially with access through only one side of a patient's chest required to perform all procedures.

### FIELD OF THE INVENTION

The field of the present invention is apparatus and methods for performing minimally invasive surgery, more particularly to ablation procedures performed with minimally invasive surgical techniques and apparatus.

### BACKGROUND OF THE INVENTION

Various medical conditions, diseases and dysfunctions may be treated by ablation, using various ablation devices and techniques. Ablation is generally carried out to kill or destroy tissue at the site of treatment to bring about an improvement in the medical condition being treated.

In the cardiac field, cardiac arrhythmias, and particularly atrial fibrillation are conditions that have been treated with some success by various procedures using many different types of ablation technologies. Atrial fibrillation continues to be one of the most persistent and common of the cardiac arrhythmias, and may further be associated with other cardiovascular conditions such as stroke, congestive heart failure, cardiac arrest, and/or hypertensive cardiovascular disease, among others. Left untreated, serious consequences may result from atrial fibrillation, whether or not associated with the other conditions mentioned, including reduced cardiac output and other hemodynamic consequences due to a loss of coordination and synchronicity of the beating of the atria and the ventricles, possible irregular ventricular rhythm, atrioventricular valve regurgitation, and increased risk of thromboembolism and stroke.

As mentioned, various procedures and technologies have been applied to the treatment of atrial arrhythmias/fibrillation. Drug treatment is often the first approach to treatment, where it is attempted to maintain normal sinus rhythm and/or decrease ventricular rhythm. However, drug treatment is often not sufficiently effective and further measures must be taken to control the arrhythmia.

Electrical cardioversion and sometimes chemical cardioversion have been used, with less than satisfactory results, particularly with regard to restoring normal cardiac rhythms and the normal hemodynamics associated with such.

A surgical procedure known as the MAZE III (which evolved from the original MAZE procedure) procedure involves electrophysiological mapping of the atria to identifying macroreentrant circuits, and then breaking up the identified circuits (thought to be the drivers of the fibrillation) by surgically cutting or burning a maze pattern in the atrium to prevent the reentrant circuits from being able to conduct therethrough. The prevention of the reentrant circuits allows sinus impulses to activate the atrial myocardium without interference by reentering conduction circuits, thereby preventing fibrillation. This procedure has been shown to be effective, but generally requires the use of cardiopulmonary bypass, and is a highly invasive procedure associated with high morbidity.

Other procedures have been developed to perform transmural ablation of the heart wall or adjacent tissue walls. Transmural ablation may be grouped into two main categories of procedures: endocardial and epicardial. Endocardial procedures are performed from inside the wall (typically the myocardium) that is to be ablated, and is generally carried out by delivering one or more ablation devices into the chambers of the heart by catheter delivery, typically through the arteries and/or veins of the patient. Epicardial procedures are performed from the outside wall (typically the myocardium) of the tissue that is to be ablated, often using devices that are introduced through the chest and between the pericardium and the tissue to be ablated. However, mapping may still be required to determine where to apply an epicardial device, which may be accomplished using one or more instruments endocardially, or epicardial mapping may be performed. Various types of ablation devices are provided for both endocardial and epicardial procedures, including radiofrequency (RF), microwave, ultrasound, heated fluids, cryogenics and laser. Epicardial ablation techniques provide the distinct advantage that they may be performed on the beating heart without the use of cardiopulmonary bypass.

When performing procedures to treat atrial fibrillation, an important aspect of the procedure generally is to isolate the pulmonary veins from the surrounding myocardium. The pulmonary veins connect the lungs to the left atrium of the heart, and join the left atrial wall on the posterior side of the heart. When performing open chest cardiac surgery, such as facilitated by a full sternotomy, for example, epicardial ablation may be readily performed to create the requisite lesions for isolation of the pulmonary veins from the surrounding myocardium. Treatment of atrial ablation by open chest procedures, without performing other cardiac surgeries in tandem, has been limited by the substantial complexity and morbidity of the procedure. However, for less invasive procedures, the location of the pulmonary veins creates significant difficulties, as typically one or more lesions are required to be formed to completely encircle these veins.

One example of a less invasive surgical procedure for atrial fibrillation has been reported by Saltman, “A Completely Endoscopic Approach to Microwave Ablation for Atrial Fibrillation”, The Heart Surgery Forum, #2003-11333 6 (3), 2003, which is incorporated herein in its entirety, by reference thereto. In carrying out this procedure, the patient is placed on double lumen endotracheal anesthesia and the right lung is initially deflated. Three ports (5 mm port in fifth intercostal space, 5 mm port in fourth intercostal space, and a 10 mm port in the sixth intercostal space) are created through the right chest of the patient, and the pericardium is then dissected to enable two catheters to be placed, one into the transverse sinus and one into the oblique sinus. Instruments are removed from the right chest, and the right lung is re-inflated. Next, the left lung is deflated, and a mirror reflection of the port pattern on the right chest is created through the left chest. The pericardium on the left side is dissected to expose the left atrial appendage and the two catheters having been initially inserted from the right side are retrieved and pulled through one of the left side ports. The two catheter ends are then tied and/or sutured together and are reinserted through the same left side port and into the left chest. The leader of a Flex 10 microwave probe (Guidant Corporation, Santa Clara, Calif.) is sutured to the end of the upper catheter on the right hand side of the patient, and the lower catheter is pulled out of a right side port to pull the Flex 10 into the right chest and lead it around the pulmonary veins. Once in proper position, the Flex 10 is incrementally actuated to form a lesion around the pulmonary veins. The remaining catheter and Flex 10 are then pulled out of the chest and follow-up steps are carried out to close the ports in the patient and complete the surgery.

Although advances have been made to reduce the morbidity of atrial ablation procedures, as noted above, there remains a continuing need for devices, techniques, systems and procedures to further reduce the invasiveness of such procedures, thereby reducing morbidity, as well as potentially reducing the amount of time required for a patient to be in surgery, as well as reducing recovery time.

### SUMMARY OF THE INVENTION

In accordance with the present invention, apparatus and methods for performing endoscopic surgical procedures are provided where only a minimal number of (or even one) openings are required to perform the procedures. Ablation procedures, including epicardial ablation procedures and apparatus for performing such procedures are described. Epicardial atrial ablation may be performed epicardially with access through only one side of a patient's chest required to perform all procedures.

An endoscopic procedure requiring access through only one side of a patient's chest is provided, including advancing an instrument through an opening in the right chest of the patient; dissecting the patient's pericardium to provide access to a transverse pericardial sinus; advancing a lead through the pericardium and the transverse pericardial sinus and into an oblique pericardial sinus of the patient; dissecting the patient's pericardium to provide access to the oblique pericardial sinus; inserting an instrument into the oblique pericardial sinus; and connecting the lead and the instrument together in the oblique pericardial sinus.

A minimally invasive method of encircling the pulmonary veins of a patient, is provided, wherein entry into only the one side of the patient is required, including the steps of: advancing a lead through an opening in the chest of the patient, through a first opening in the pericardium, and into a transverse pericardial sinus of the patient, across the transverse pericardial sinus and into an oblique pericardial sinus of the patient as the lead tracks downward along a closed border of the pericardium on a side of the heart opposite to the opening in the pericardium; inserting an instrument through a second opening in the pericardium and into the oblique pericardial sinus; and connecting the lead and the instrument together in the oblique pericardial sinus.

Surgical apparatus are provided. In one embodiment, surgical apparatus include an elongated body having distal and proximal end portions and a lumen therethrough; a lens in the lumen; a transparent tip extending distally from the distal end portion; and an elongated tube slidable over the elongated body and adapted to cannulate an opening through tissue formed by the tip.

In another embodiment surgical apparatus are provided including an endoscope having an elongated body and a lumen therethrough, a lens in the lumen, a distal end portion of the endoscope including a distal end portion of the elongated body and a tip extending distally from the elongated body, wherein images are viewable through the tip, lens and lumen; and an elongated lead cinched over the distal end portion of the endoscope and extending proximally therefrom.

In a still further embodiment, surgical apparatus include a dissecting endoscope having an elongated body having distal and proximal end portions and a lumen therethrough; a lens in the lumen, and a transparent tip extending distally from the distal end portion; and a tube having an inside diameter larger than an outside diameter of the elongated body, and slidable over the elongated body, the tube being adapted to be mounted on the elongated body prior to dissecting an opening through tissue by the dissecting endoscope, and to be slid distally with respect to the elongated body and through an opening established by the dissecting endoscope, thereby cannulating the opening, even after removal of the dissecting endoscope therefrom.

In another embodiment, surgical apparatus include an elongated lead having sufficient length to extend from an opening in a right chest of a patient and out of the patient's body at a proximal end of the lead, around four pulmonary veins of the patient and back out of the opening in the right side of the patient at a distal end of the elongated lead, with the elongated lead further comprising a first connector at a distal end thereof; and an elongated instrument having sufficient length to extend through the opening, or a second opening in the right side of the patient to connect with the lead in an oblique pericardial sinus of the patient, the elongated instrument further comprising a second connector, wherein a connection between the elongated instrument and the elongated lead is made via the first and second connectors.

A surgical instrument for performing endoscopic functions is provided, including an elongated body having distal and proximal end portions and a lumen therethrough; a lens in the lumen; and a transparent tip extending distally from the distal end portion, said transparent tip having a distal end portion having a first cross-sectional area larger than a second cross-sectional area of the tip at a location proximal of the distal end portion of the tip, wherein images are viewable through the elongated body, lens and transparent tip.

A surgical device is described, including an elongated tube having sufficient length to extend from an opening in a right chest of a patient and out of the patient's body at a proximal end of the tube, around four pulmonary veins of the patient and back out of the opening in the right side of the patient at a distal end of the tube, the tube further comprising at least one slit between proximal and distal ends of the tube, with the at least one slit being sufficiently long to form an opening to slidably receive an endoscope.

Surgical apparatus are provided including an elongated lead having sufficient length to extend from an opening in a right chest of a patient and out of the patient's body at a proximal end of the lead, around four pulmonary veins of the patient and back out of the opening in the right side of the patient at a distal end of the elongated lead, the elongated lead comprising a first tube and a second tube, a proximal end of the first tube being connectable with a distal end of the second tube; and an elongated instrument having sufficient length to extend through the opening and into a transverse pericardial sinus of the patient, wherein the first tube is configured to be freely slidable over the elongated instrument, and the first tube has a length less than a length of the elongated instrument, so that a distal end of the elongated instrument extends beyond a distal end of the first tube when the first tube is slid over the elongated instrument.

In another embodiment, surgical apparatus include an expandable length tube having an expanded length configuration having sufficient length to extend from an opening in a right chest of a patient through a transverse pericardial sinus of the patient, around left pulmonary veins of the patient and into an oblique pericardial sinus of the patient, and a compressed length configuration having a length shorter than the expandable length configuration, the tube having an opening therethrough configured to pass an ablation probe therethrough.

A surgical device is provided, including an elongated tubular instrument having a main tube with sufficient length to extend through an opening in the chest wall of a patient, through the pericardium of the patient and into the transverse pericardial sinus while a proximal portion of the instrument remains outside the body of the patient; a transparent distal tip mounted to a distal end of the main tube; and a second tube running parallel to the main tube and having an inside diameter sufficient to pass a snare catheter therethrough.

In another embodiment, a surgical device includes an elongated tubular instrument having a main tube with sufficient length to extend through an opening in the chest wall of a patient, through the pericardium of the patient and into the transverse pericardial sinus while a proximal portion of said instrument remains outside the body of the patient; a transparent distal tip mounted to a distal end of said main tube; a lumen extending through said main tube and opening to a proximal end of said device, said lumen configured to receive an endoscope to permit viewing by said endoscope through said transparent distal tip; and a second lumen extending through said main tube and opening at a proximal end portion of said device to receive a snare catheter therethrough.

In still another embodiment, a surgical device includes an elongated ablation probe having a proximal end portion and a distal end portion; and an elongated, flexible tube having a proximal end portion and a distal end portion, wherein the proximal end portion of the tube is releasably connectable to the distal end portion of the probe.

These and other advantages and features of the invention will become apparent to those persons skilled in the art upon reading the details of the methods, apparatus and devices as more fully described below.

### BRIEF DESCRIPTION OF THE DRAWINGS

FIG. 1A shows an assembly of devices that may be used during ablation procedures according to the present invention.

FIG. 1B is an enlarged, schematic view of the tip of the dissecting endoscope shown in FIG. 1A.

FIG. 2A illustrates a cutaway anterior view of a human heart with a dissecting instrument being used to penetrate the pericardium.

FIG. 2B illustrates an opening in the pericardium having been cannulated by a tube, and a snare catheter having been advanced through the tube and routed through the transverse pericardial sinus and into the oblique pericardial sinus, thereby partially surrounding the pulmonary veins.

FIG. 2C illustrates insertion of an instrument into the oblique pericardial sinus to be connected with the snare catheter.

FIG. 2D illustrates drawing the instrument (shown in FIG. 2D) out of the body, which in turn begins to pull the snare catheter out of the body and to draw a connected ablation probe into the body along a desired pathway.

FIG. 3A is an illustration of one example of a snare catheter.

FIG. 3B is an illustration of another example of a snare catheter.

FIG. 4A shows an operating endoscope that may be used in at least one embodiment of the present invention.

FIG. 4B is a partial view showing endoscopic shears being extended through the operating endoscope shown in FIG. 4A to perform incision of the pleural and pericardial layers.

FIG. 4C shows attachment of a snare catheter to an instrument by snaring the instrument while in the location of the oblique pericardial sinus.

FIG. 4D illustrates drawing the snare catheter out of the body by pulling the attached instrument out of the body.

FIGS. 5A, 5B and 5C illustrate alternative, removable tips that may be used on an endoscope according to the present invention.

FIGS. 6A, 6B, 6C and 6D illustrate alternative constructs for connecting a catheter with an endoscope for purposes of drawing the catheter out of the oblique pericardial sinus, with progressive views illustrating a technique for making the connection.

FIG. 7A schematically illustrates another embodiment of a snare catheter.

FIG. 7B illustrates one manner in which an ablation probe may be connected to a snare catheter.

FIGS. 8A and 8B illustrate alternative embodiments of a tube that may be used in carrying out procedures according to the present invention.

FIG. 8C illustrates use of one of the tubes shown in FIGS. 8A and 8B.

FIG. 8D shows a cross sectional view of the tube of FIG. 8C, taken along line 8-8.

FIGS. 9A, 9B, 9C, 9D, 9E and 9F illustrate various aspects of another variation of devices that may be used to carry out at least one embodiment of the present methods.

FIGS. 10A, 10B, 10C and 10D illustrate various aspects of still another variation of devices that may be used to carry out at least one embodiment of the present methods.

FIG. 11 illustrates another example of a dissecting endoscope according to the present invention.

FIGS. 12A, 12B, 12C and 12D illustrate an ablation probe with a connected tube and its use.

FIGS. 13A-13C are various examples of alternative endoscope devices according to the present invention.

FIG. 13D is an exploded view of the device shown in FIG. 13A.

FIG. 14A is a partial, sectional, schematic view of an endoscope.

FIG. 14B schematically shows positioning of a tip lens relative to the light emitting fibers in the distal tip of an endoscope.

FIG. 14C shows a variation of a tip lens and positional arrangement relative to light emitting fibers in the distal tip of an endoscope.

### DETAILED DESCRIPTION OF THE INVENTION

Before the present methods and devices are described, it is to be understood that this invention is not limited to particular surgeries, tools, materials, methods or devices described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an activation” includes a plurality of such activations and reference to “the lesion” includes reference to one or more lesions and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “open-chest procedure” refers to a surgical procedure wherein access for performing the procedure is provided by a full sternotomy or thoracotomy, a sternotomy wherein the sternum is incised and the cut sternum is separated using a sternal retractor, or a thoracotomy wherein an incision is performed between a patient's ribs and the incision between the ribs is separated using a retractor to open the chest cavity for access thereto.

The term “closed-chest procedure” or “minimally invasive procedure” refers to a surgical procedure wherein access for performing the procedure is provided by one or more openings which are much smaller than the opening provided by an open-chest procedure, and wherein a traditional sternotomy is not performed. Closed-chest or minimally invasive procedures may include those where access is provided by any of a number of different approaches, including mini-sternotomy, thoracotomy or mini-thoracotomy, or less invasively through a port provided within the chest cavity of the patient, e.g., between the ribs or in a subxyphoid area, with or without the visual assistance of a thoracoscope.

The term “reduced-access surgical site” refers to a surgical site or operating space that has not been opened fully to the environment for access by a surgeon. Thus, for example, closed-chest procedures are carried out in reduced-access surgical sites. Other procedures, including procedures outside of the chest cavity, such as in the abdominal cavity or other locations of the body, may be carried out as reduced access procedures in reduced-access surgical sites. For example, the surgical site may be accessed through one or more ports, cannulae, or other small opening(s). What is often referred to as endoscopic surgery is surgery carried out in a reduced-access surgical site.

Conventional minimally invasive thoracoscopy surgery typically uses three ports on each side of the patient from which access is required. A camera (e.g., an endoscope) is inserted through one port, typically the central port to give the surgeon a “bird's eye” or “god's eye” view of the surgical target. Instruments (e.g., graspers, scissors or other instruments) may then be inserted through the other two ports (e.g., on opposite sides of the camera) and manipulated to perform a surgical procedure, as the working ends of the instruments are viewed via the camera. For example, graspers may be inserted through one of the other two ports and a Kitner sponge stick may be inserted through the other of the two ports. This type of procedure also takes at least two people to perform it: typically an assistant will hold and operate the endoscope through the central port, while a surgeon manipulates the tools through the other two ports. For example, the surgeon may lift up the vena cava with one instrument, and then use the sponge stick to perform the dissection of pericardial layers. As the dissection progresses further inwardly, it can no longer be seen by the endoscope where it is originally positioned. When moving the endoscope in closer to regain a view of the dissection, there is risk of contacting the lens of the scope with the vena cava or other tissue, which blurs the view. Accordingly, the endoscope must then be taken all the way back out of the body through the central port, and wiped off or otherwise cleaned and reinserted. However, the same risk of smudging or blurring the lens persists each time the endoscope needs to be further advanced into the operative site. Accordingly, such a procedure is man-hour intensive, requiring at least two operators, and time consuming, as well as difficult. The present endoscopes use tips that are self-cleaning, and provide a direct view of the surgical procedure that is being performed, while at the same time, being controllable by the surgeon that is also performing the surgical procedure.

With regard to thoracoscopic endocardial atrial ablation procedures, some current surgical techniques may take in the neighborhood of three hours just to accomplish the task of encircling the pulmonary veins in preparation for performing an epicardial ablation.

The present invention provides simple, reliable and safe techniques for minimally invasive procedures, such as closed-chest cardiac procedures that require ports (typically three or less) on only one side of the patient, thereby reducing the invasiveness of procedures that typically require ports on both sides of the patient. Further, the present techniques are much faster, typically requiring only minutes (e.g., about thirty to sixty minutes), as opposed to hours (e.g., about three hours) to encircle the pulmonary veins, for example. Even for procedures that typically are single sided, the present invention may reduce the number of ports that are required on one side of the patient, compared to the three previously required by conventional techniques. Not only are the present techniques less invasive, but devices provided make the procedures easier and safer to carry out.

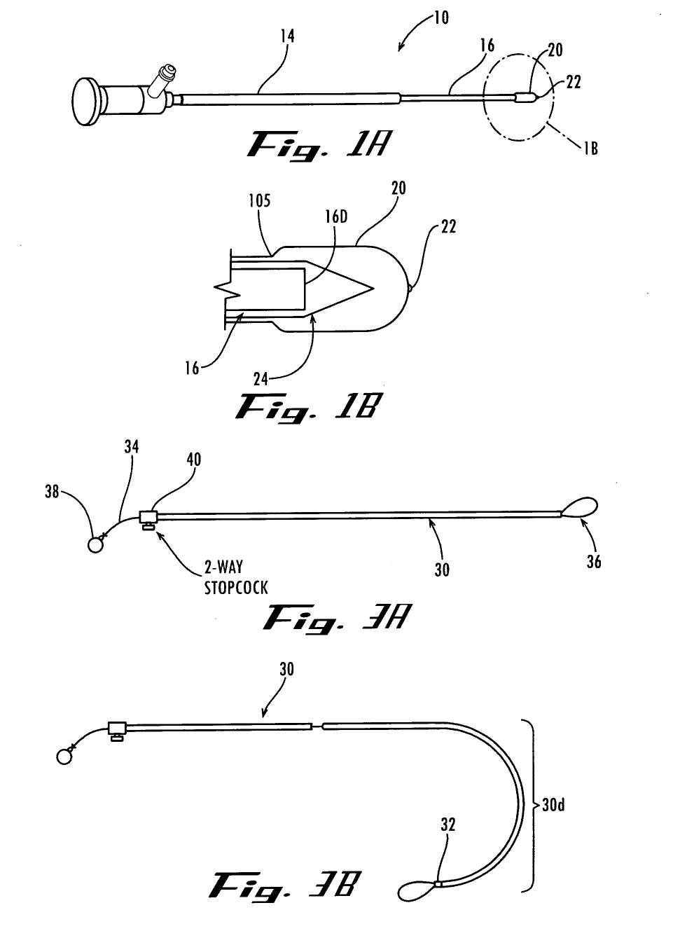

Referring now to FIG. 1, a dissecting instrument 10 is shown that may be used to carry out procedures during the performance of methods described herein. Dissecting instrument 10 includes an endoscope having an elongated tube or shaft 16 (e.g., a rigid tube/telescope having a diameter of about 5 to about 7 mm and length of about 25-40 cm). Such endoscopes are available from various companies, including Olympus (Japan), and Stortz and Scholly (Germany). Tube or shaft 16 is typically rigid to provide the best maneuverability, once instrument 10 has been inserted into an area to perform surgical techniques, for dissecting using tip 20. As the dissection can be viewed using the endoscope of the same instrument 10, only one opening, such as a thoracotomy or port, or other small opening to permit the insertion of instrument 10 is required for performing dissection.

For purposes of maintaining an established pathway through tissue, such as may be established by dissection as described, a non-collapsing, flexible or rigid tube 14 may be placed coaxially over the endoscope shaft 16 as shown in FIG. 1. Tube 14 may be made from flexible material such as polyvinyl chloride or polyethylene, incorporated with metal (e.g., stainless steel, NITINOL™ (nickel-titanium alloy) or the like) or plastic (nylon, polyester, or the like) mesh to render it non-collapsing; or tube 14 may be constructed of rigid plastic, such as polycarbonate, liquid crystal plastic (LCP), ULTEM® (amorphous thermoplastic polyetherimide), or the like, or from stainless steel or the like. Tube 14 is freely slidable over shaft 16 and is initially positioned over the proximal portion of shaft 16 as shown in FIG. 1, thereby leaving a distal portion with a smaller diameter profile for better mobility around the surgical space during dissection. For example, tube 14 may be about two thirds the length of shaft 16 for use in non-invasive epicardial ablation techniques as described below, wherein tube is about 27 cm. Of course, the present invention is not limited to this length or to the proportion of the lengths of tube 14 to shaft 16, as these may vary depending upon the applications that the instrument 10 may be used for, as well as other factors.

Tip 20 is transparent and generally blunt and may be of a generally spherical or other blunt curvature. However, a small (e.g., about 1 mm diameter) nipple or protrusion 22 may be provided to extend from the distal end of tip 20 to increase friction with the tip 20 against tissue to facilitate dissection. Tip 20 is transparent to enable direct viewing to the surgical site through endoscope 16 and of the dissection as it is proceeding. Tip 20 is distanced from the lens at the distal end 16d of endoscope shaft 16 so that any tissue that contacts tip 20 can still be viewed by the endoscope, as the endoscope lens does not become smeared or blurred. Also, the distance between the external distal surface of tip 20 and the lens at the distal end of shaft 16d permits a field of view by endoscope 10, so that the anatomy can be better discerned since all tissue in contact with the length or long axis of the tip is viewed, rather than having a view that is limited to tissue that the endoscope lens contacts, as is the case when using a standard endoscope arrangement. For example, without a tip, an endoscope may bump up against the vena cava, but the view will not permit identification of such, as a constant wall of tissue will be seen in the field of view. Using a tip, however, a length of the vessel will be seen, with some surrounding background in the field of view, so that the vessel can be identified as such. Tip 20 may be removable to allow interchanging tip 20 with another tip for carrying out another function, as will be described in more detail below. Optionally, a tapered or conical transparent tip 24 may be mounted concentrically within with respect to the endoscope and tip 20. The surface of angled or conical tip 24 breaks up the reflected waves from the blunt tip 20 and prevents the formation of a ring of reflected light in the visualization through endoscope 16 that might otherwise occur. Further details about such an arrangement are described in co-pending application Ser. No. (application Ser. No. not yet assigned, Attorney's Docket No. GUID-068) filed concurrently herewith (i.e., May, 26, 2005) and titled “Ablation Instruments and Methods for Performing Ablation”, which is incorporated herein, in its entirety, by reference thereto. This configuration of a sharper tip 20 within a blunt tip 20 may be employed in ablation devices 10 that use a blunt tip 20 as described above, as well as other instruments designed to contact tissues while providing visualization.

A light emitter (not shown) may be provided in the distal end portion of instrument 10 to direct light out of the distal end so that the operator may visualize the position of the distal end in the surgical site by viewing through the endoscope 16. Like some existing endoscopes, the endoscope 16 provided with instrument 10 contains a visualization portion (e.g., rod lenses) and a fiber optic light-carrying portion (e.g., optical transmission fibers). A light cable connects to endoscope 16 and supplies light to the light-carrying portion, from an external light source (e.g., Xenon light source, which may be in the vicinity of 300 Watts power). Thus, a surgeon or operator may directly view the positioning and movements of the distal end of instrument 10 from outside the patient, without the need to resort to any indirect visualization or sensing techniques for positioning, and this greatly increases the accuracy and precision of placement of instrument 10 for performing dissection. The fact that the procedure can be viewed through the same instrument that is carrying out the dissection also removes the requirement for placing an additional opening through the patient to insert a separate endoscope, as is done with traditional endoscopic surgeries. A power supply line (not shown) may be connected to the light source to extend proximally out of the instrument 10 to be connected to an external power source.

While typically rigid, the distal end portion of instrument 10 may be formed to be articulating, to provide a greater range of motion during dissecting as well as for directing placement of tube 14 in examples where tube 14 is flexible. Further alternatively, endoscope 16 may be made flexible or malleable for situations where it would be advantageous for the particular application or technique being practiced.

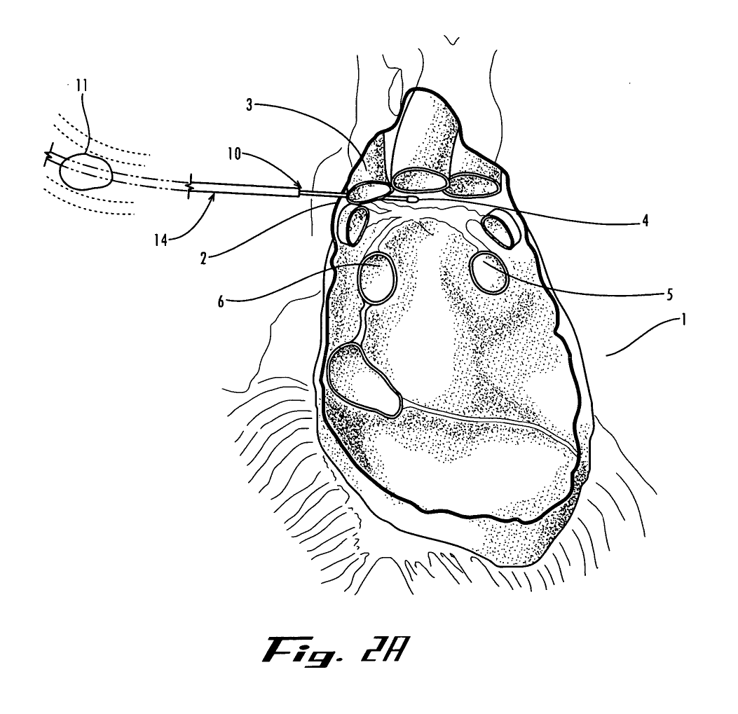

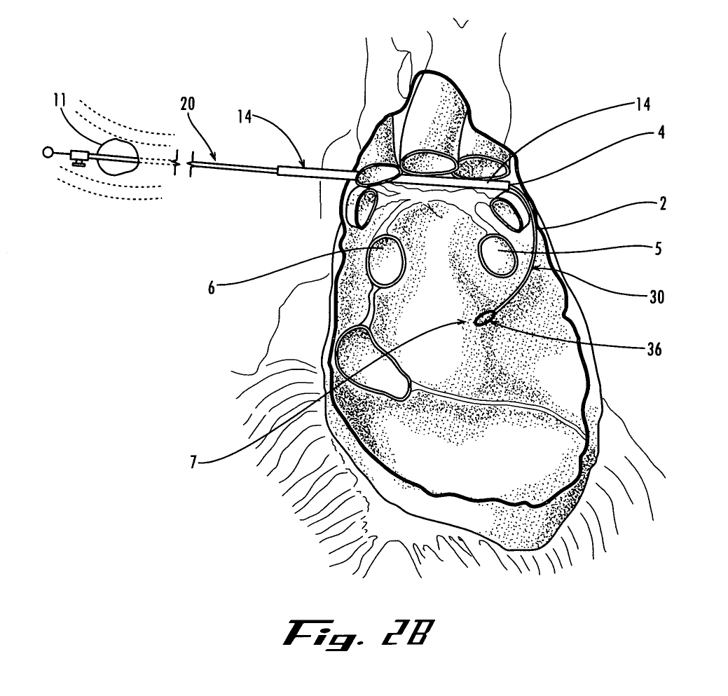

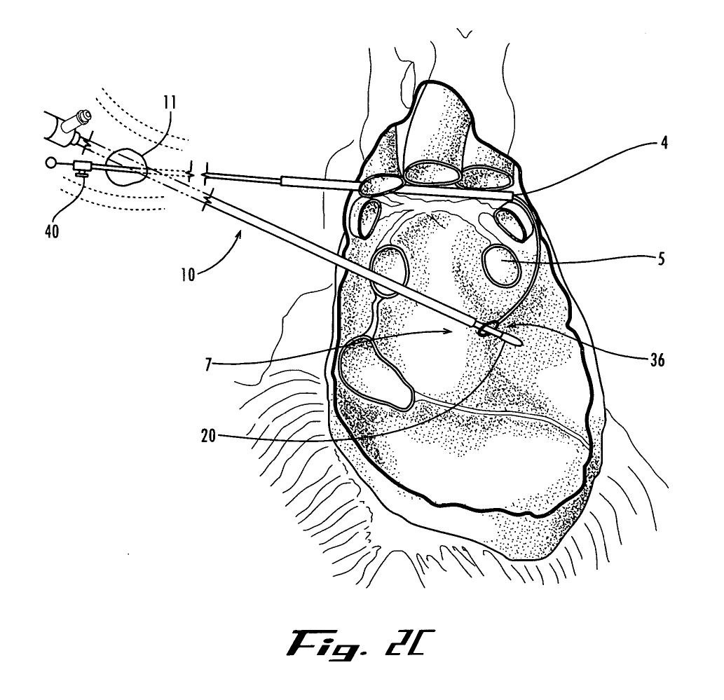

An example of using instrument 10 in a method according to the present invention will now be described, initially with reference to FIG. 2A. FIG. 2A illustrates a cutaway anterior view of a human heart 1 with instrument 10 being used to penetrate the pericardial reflection 2. The right side of the pericardium has been previously incised, using endoscopic shears (not shown). At least one port or opening 11 is formed in the right chest of the patient (e.g., a port 111 though the third intercostal space of the right chest) to provide access to the heart by instrument 10. Instrument 10, along with tube 14 is next inserted through opening 11 and tip 20,22 is used to dissect through pericardium 2 until superior vena cava 3 can be visualized through endoscope 16. Dissection may be performed by carefully scraping tip 20/protrusion 22 against the pericardial tissue to separate it with a side-to-side or up-and-down motion of tip 20, for example. Dissection through the pericardial membrane (pericardial reflection) is made posterior to the superior vena cava thereby providing an entrance to the transverse pericardial sinus 4. Upon achieving access to the transverse pericardial sinus 4 with instrument 10, sleeve or tube 14 may then be advanced distally along instrument 10 to insert tube 14 into the transverse pericardial sinus. Although the anatomical structures described herein are well-known and would be readily understood by those of ordinary skill in the art reading the present disclosure and referring to the Figs. herein, additional views may be found in United States Application Publication No. US2004/0111101 A1, (e.g., see FIG. 1 and description thereof), which published on Jun. 10, 2004 and which is hereby incorporated herein, in its entirety, by reference thereto. Tube 14 may continue to be advanced until the distal end of tube nearly traverses the width of the heart and is near the left pulmonary veins 5 as shown in FIG. 2B. Instrument 10 may be removed, leaving tube 14 in place, thereby cannulating the transverse sinus.

A snare catheter 30 may next be inserted into tube 14 and manipulated around the pulmonary veins as described below. Snare catheter 30 may be constructed of flexible plastic material such as polyethylene, polytetrafluoroethylene (PTFE, e.g., TEFLON®), polyvinyl chloride, nylon, or the like. Snare catheter 30 may be formed to be substantially straight in an unstressed state (FIG. 3A) or to have a preconfigured bend in its distal section 30d as shown in FIG. 3B, (e.g., of about the last 10-15 cm of catheter length, which may assist in maneuvering the catheter along a similar curved pathway within the body, such as directing the tip downward after it has been passed through tube 14. Catheter 30 is sufficiently small to be easily slid through tube 14 and may be on the order of about 6 Fr in diameter, for example. Catheter 30 may be provided with a rigid distal tip 32 made from a biocompatible metal or rigid polymer. Rigid tip 32 allows the snare to hold the ball tip securely, as it does not give as the ball tip is drawn against it, wherein a soft tip may allow the ball tip to slip out when traction is applied to the snare catheter.

Catheter 30 is tubular, to allow suture line or wire 34 to pass therethrough. Suture line 34 includes a suture loop 36 formed with a sliding knot (an Endoloop) in a distal end thereof. Suture loop 36 is located distally of the distal end of catheter 30. Suture loop may be formed from a conventional suture material or braided stainless steel wire cable, for example. Alternatively, the entire suture line may be made of NITINOL®, or other nickel-titanium alloy without the need to use a sliding knot. The proximal end of suture line or wire 34 (or tail of the suture loop) extends through catheter 30 and proximally out of the proximal end of catheter 30, where it may be attached to a pull tab 38. Further, a lock 40 such as a two-way stopcock, clamp, hemostats, or other surgical clamp, tool or locking mechanism may be provided to grasp suture line or wire 34 and abut the proximal end of catheter 30 to prevent backsliding of catheter 30 with respect to suture line 34 (i.e., sliding of catheter 30 proximally with respect to suture line 34) as this device is used to draw an ablation device into position, as will be described below.

As catheter 30 is inserted through tube 14, once the distal end of catheter is pushed out the distal end of tube 14 and against the pericardium 2 on the left side of the heart, the distal end of catheter 30 and suture loop 36 are deflected downwardly and are further advanced, into the oblique pericardial sinus 7, which is a majority of the region shown just beneath the left 5 and right 6 pulmonary veins on the posterior aspect of the heart in FIG. 2B. As can be seen in FIG. 2B, catheter 30 at this stage has begun to encircle the pulmonary veins 5,6.

Next, with catheter 30 remaining in place as shown in FIG. 2B, instrument 10 may be reinserted through opening 11 and used to dissect the pericardium at a location posterior to the inferior vena cava to form an opening to the oblique pericardial sinus. Instrument 10 may then be inserted into the oblique pericardial sinus 7 as shown in FIG. 2C, while viewing through the endoscope 16 to align tip 20 with suture loop 36. Upon successfully passing tip 20 through suture loop 36 as shown in FIG. 2C, the operator next applies traction to suture line 34, while holding catheter 30 stationary with respect to movement of the suture line 34. This causes suture loop 36 to cinch down as suture line 34 is pulled through the sliding knot of the suture loop 36. If the loop 36 is formed of NITINOL®, or other nickel-titanium alloy, no sliding knot is present, rather the loop diameter decreases by virtue of the loop being pulled into the catheter. This action is continued until suture loop 36 is in tight contact with device 10 proximal of tip 20, thereby effectively “lassoing” instrument 10. Note that since instrument 10 necks down just proximal of tip 20 as shown in FIGS. 1A and 1B, that suture loop is capable of maintaining a grip on device 10, even under tension. Lock 40 is fixed to suture line or wire 34 in a position abutting the proximal end of catheter 30 to prevent catheter 30 from backsliding, as noted above, and particularly to prevent suture loop 36 from expanding.

Once instrument 10 has been captured by suture loop 36, as described, an ablation device 50 is fixed to the proximal end of suture line 34 (FIG. 2D), after removing pull tab 38 (if used) and lock 40, such as by severing suture line 34 distally of those features. As one method, suture line 34 may be tied to a distal leader 52 of ablation device 50. A suitable ablation device that may be used as ablation device 50 is the Flex 10 microwave probe (Guidant Corporation, Santa Clara, Calif.), although the present invention is not limited to use of this product only. Other ablation devices configured to form a long linear lesion and which are sufficiently flexible to surround the pulmonary veins as described herein may be substituted. Further, the energy type for performing the ablation need not be microwave energy, but may alternatively be any of the other types of energy that have been used to form lesions (e.g., Rf, electrical, heat, chemical, ultrasonic, etc.).

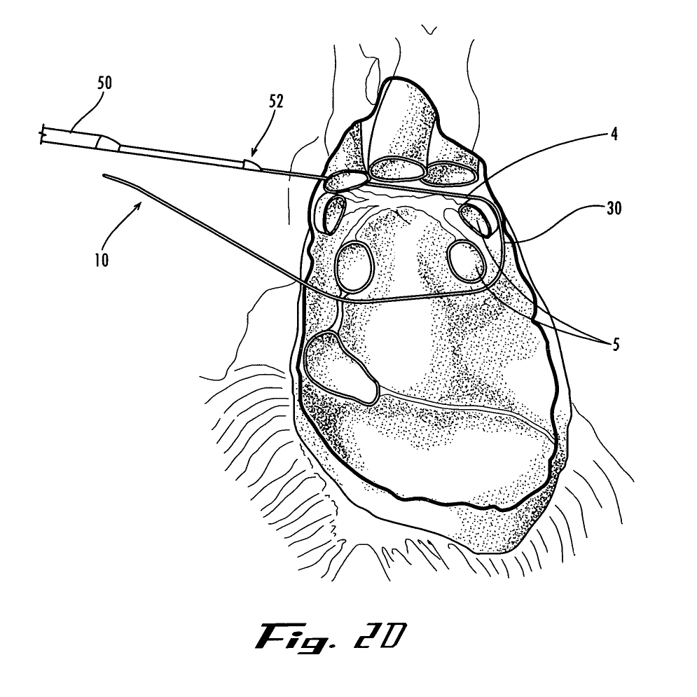

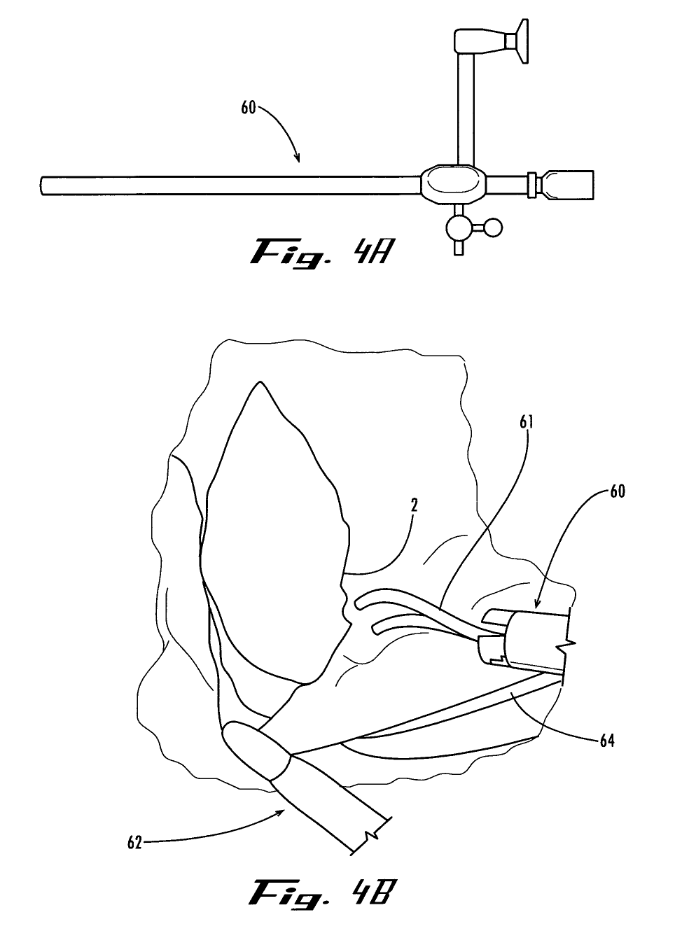

By pulling instrument 10 through the opening 11, this begins to draw catheter 30 out of the opening 11 along with instrument 10 as long as suture loop maintains the capture of tip 20. Movement of catheter 30, in turn, draws ablation device 50 in through opening 11 and leads ablation device 50 around the pulmonary veins into the position previously occupied by catheter 30. By drawing catheter 30 through the same opening 11 that ablation device 50 was drawn into, this maneuvers ablation device 50 to surround the pulmonary veins, as shown in FIG. 2D. Epicardial ablation probe 50 is then detached from catheter 30, and endoscopic graspers 62 in operating endoscope 60 (e.g., see FIGS. 4A,4B) may be used to grasp the distal portion of ablation probe 50 to insert it back into the mediastinum. Epicardial ablation probe 50 is positioned to completely encircle the four pulmonary veins, and may be held in position using graspers 62 while energy is being delivered to accomplish the epicardial ablation. Once in proper position, ablation device 50 is actuated to form a lesion to surround the pulmonary veins. When using the Flex 10 or similar product, ablation device 50 may be incrementally actuated to form the lesion around the pulmonary veins, a segment at a time. The remaining portion of catheter 30 and ablation device 50 are then pulled out of the chest and follow-up steps are carried out to close the opening in the patient and complete the surgery.

Alternative to the single port method described above, a similar method may be carried out through the use of two ports. While this alternative procedure requires some additional manipulation, it is a viable alternative and may be used, for example, if the surgeon wants to use additional tools for carrying out any of the procedures of the method. For example, the surgeon may want to use additional tools to assist in initially opening the pericardium. Additionally, some surgeons will be more comfortable or accustomed to using multiple ports to provide additional instruments (e.g., graspers) to the surgical site to retract tissue and thereby increase exposure of the anatomy, for example. For example, port 11 may be placed in the second intercostal space, in the anterior to mid-axillary line, and the second port 13 may be placed in the third or fourth intercostal space, in the anterior to mid-axillary line. The superior port 11 may be approximately 10 to 15 mm in diameter, and the inferior port 13 may be approximately 5 to 12 mm in diameter, but is typically about 5 mm in diameter.

An operating endoscope 60 (e.g., a 12 mm, 0-degree operating endoscope) with endoscopic graspers 62 advanced through the working channel, may be inserted through opening 11 and used to grasp the right pleura and pericardium 2 anterior to the phrenic nerve 64, for example, as shown in FIGS. 4A and 4B. Additionally, a pair of endoscopic shears 61 may be inserted through opening 13 and used to incise the pericardium from the superior vena cava extending to the inferior vena cava, while graspers 62 are used to hold and retract the pleura and pericardium to facilitate the incision using shears 61.

Dissecting endoscope 10 may then be inserted through opening 11, as shown in FIG. 2A, and used to dissect through the pericardial reflection posterior to the superior vena cava, and superior to the right superior pulmonary vein, to enter the transverse pericardial sinus 4. Dissecting endoscope 10 may be advanced through transverse sinus 4 until the distal end of dissecting endoscope 10 reaches the opening of the transverse sinus on the left border of the pericardium. The left atrial appendage is typically visible at the opening of the transverse pericardial sinus. Tube 14 is then advanced in the same manner as described with regard to the previous method, along dissecting endoscope 10, until its distal end extends beyond the distal end of dissecting endoscope 10. Dissecting endoscope 10 is then removed from tube 14 and catheter 30 is inserted into tube 14. As catheter 30 is passed through tube 14, the distal end eventually contacts the left border of the pericardial sac and tracks inferiorly into the oblique pericardial sinus 7, as has already been described, and as is illustrated in FIG. 2B. As it is passed through tube 14, the distal portion of catheter 30 is directed inferiorly, to aid in passage laterally to the left pulmonary veins 5 and downward into the oblique pericardial sinus 7, after it has traversed the transverse pericardial sinus 4.

Dissection scope 10 is then inserted through inferior port 13 and is used to dissect through the reflection of the pericardium posterior to the inferior vena cava, inferior to the right inferior pulmonary vein. As a result, tip 20 of dissection endoscope 10 lies in the oblique pericardial sinus 7. Catheter 30 and suture loop 36 are also in the oblique pericardial sinus 7. Distal tip 20 and suture loop 36 are visible via dissecting endoscope 10 and thus may be viewed from outside of the patient during this phase of the procedure. Dissection endoscope 10 is maneuvered to insert tip 20 through suture loop 36, as visually guided by the operator viewing the procedure via endoscope 10. Once tip 20 has been inserted through suture loop 36, as shown in FIG. 4C, suture loop 36 is cinched down around dissector 10 and locked with lock 40, thereby attaching catheter 30 to dissection endoscope 10.

Dissecting endoscope 10 is then pulled out of inferior port 13, bringing the suture loop 36 and the distal end of catheter 30 out of the patient's body, as well, as schematically represented in FIG. 4D. Suture loop 36 is then disconnected from endoscope 10, such as by releasing lock 40 to allow suture line 34 to slide with respect to catheter 30 so that more suture line can be taken up by suture loop 36, thereby expanding the loop so that it can be easily slid back over tip 20, thereby releasing the connection of suture line 34 with endoscope 10. Catheter 30 and suture loop 36 are then re-inserted into the right pleural cavity through opening 13.

Operating endoscope 60 may next be inserted into opening 11 and endoscopic graspers 62 may then be used to grasp catheter 30 and pull it out of opening 11. Alternatively, dissecting endoscope may be inserted into opening 11 and maneuvered to again pass tip 20 through suture loop 36, after which suture loop 36 can again be cinched down in the same manner as described earlier. Once so connected, catheter 30 can then be drawn out of opening 11 by pulling endoscope 10 back out of the opening, after which endoscope may again be disconnected from catheter 30 in the same way as discussed previously. Either technique results in both ends of catheter 30 protruding out of opening 11.

Next, ablation probe 50 is connected to the proximal end of catheter 30 in a manner as described previously, after stop 40 has been removed, such as by cutting for example. The distal end of catheter 30 is then pulled out of the patient's body, through opening 11, to pull ablation probe 50 into place around the pulmonary veins, into the configuration shown in FIG. 2D. Epicardial ablation probe 50 is then detached from catheter 30, and endoscopic graspers 62 in operating endoscope 60 may be used to grasp the distal portion of ablation probe 50 to insert it back into the mediastinum. Epicardial ablation probe 50 is positioned to completely encircle the four pulmonary veins, and may be held in position using graspers 62 while energy is being delivered to accomplish the epicardial ablation.

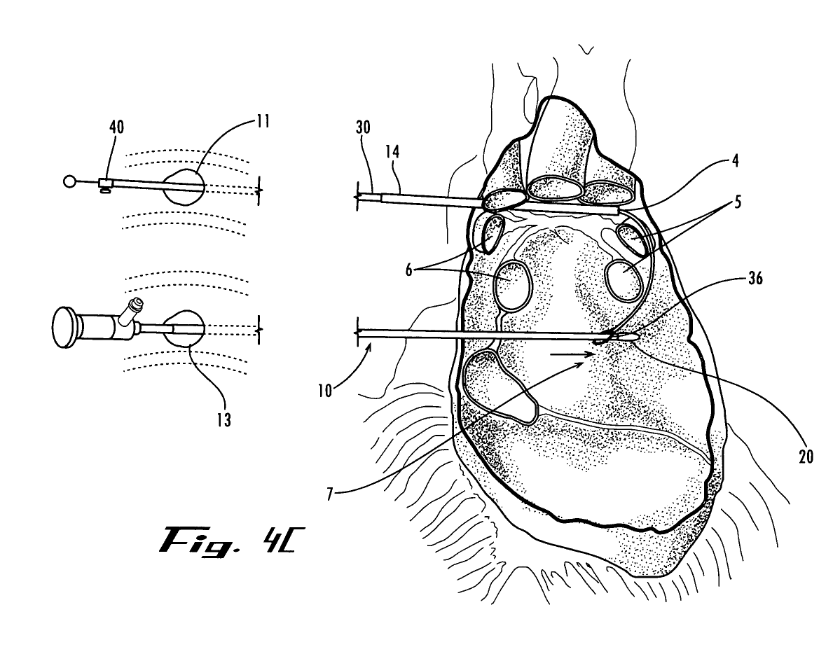

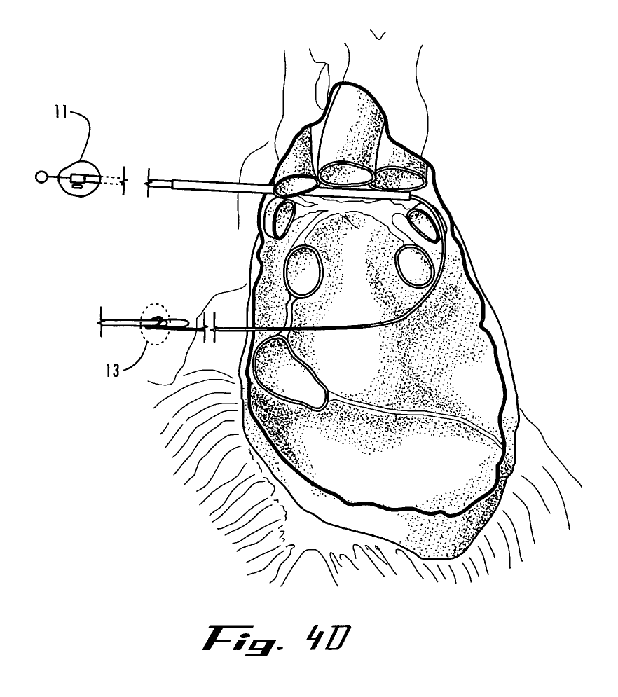

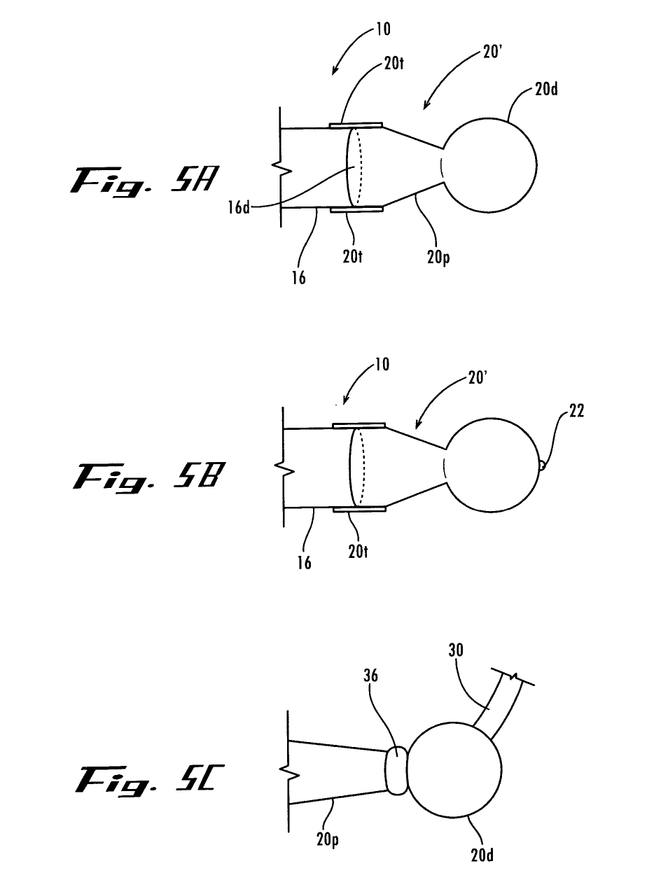

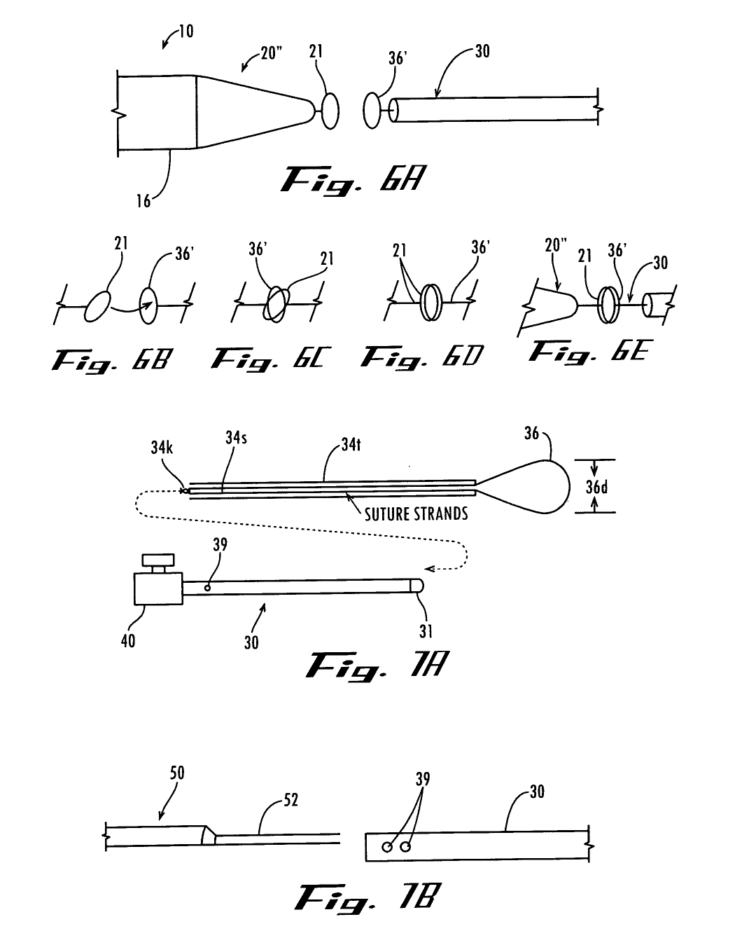

Variations of the previously described methods and devices may be employed to form a lesion surrounding the pulmonary veins by epicardial ablation. In one variation, endoscope 10 is provided with a replaceable tip 20. That is tip 20 may be removed from the distal end of endoscope and replaced with a different shape tip, such as tip 20′ shown in FIG. 5A or tip 20′ shown in FIG. 5B, or another tip having a different shape. When using endoscope 10 with tip 20 as described above, as dissecting endoscope 10 is inserted posterior to the inferior vena cava, into the oblique sinus, and manipulated for advancement through suture loop 36, this procedure may be completely visualized through endoscope 10, as noted. However, as tip 20 passes through suture loop 36, and since suture loop 36 is cinched down on dissecting endoscope 10 proximal to tip 20 at the tapered shoulder 10s formed near the proximal end of tip 20 where tip 20 meets shaft 16, the person using endoscope 10 is unable to visualize loop 36 during and after the cinching process. This is because shoulder 10s is also proximal of the distal end of the endoscope 16d where light enters the endoscope for visualization. Accordingly, monitoring of the connection between catheter 30 and endoscope 10 via suture loop 36 is not possible as endoscope 10 is pulled out of the body to in turn draw catheter 30 and ablation probe 50 along the pathway described above.

Tip 20 may be provided so as to be removable from device 10, such as by providing mating threads between tip 20 and shaft 16, for example, with appropriate sealing to prevent fluids from passing through the connection. Additionally or alternatively, tip 20 may be fixed to shaft 16 by one or more of the following: bayonet fitting, threaded stem attached to tip 20 that runs the full length of the tube 16 and is secured at the proximal end of tube 16 with a nut that mates with the threads, mating projections and holes or sockets, etc. Similarly, any tip to be interchanged with tip 20 may be provided with the same threads or other connection expedient that may be used for removably securing tip 20 to device 10 with a fluid tight seal.

Tip 20′ is provided with threads 20t at a proximal end portion thereof, for connecting tip 20′ with mating threads on the distal end of shaft 16, as shown in FIG. 5A. Tip 20′ is a ball-ending tip that includes a tapered proximal portion 20p that may be conical or some other tapering shape that reduces in cross section in a distal direction. At the distal-most portion of proximal portion 20p, where proximal portion may be smallest in cross-section, a ball-shaped or spherical distal portion 20d is integral therewith and extends distally therefrom. Spherical portion 20d may be sized on the order of about 2-4 mm in diameter, for example, typically about 3 mm. Tip 20′ may be injection molded in one piece from polycarbonate plastic, for example or from some other rigid, biocompatible and transparent plastic, glass or composite, or may be machined, for example.

In carrying out any of the previously described methods, tip 20 may be used to carry out procedures up until the time that suture loop 36 is positioned in the oblique pericardial sinus, and dissection of the pericardium at a location posterior to the inferior vena cava to form an opening to the oblique pericardial sinus has been completed. At this time, dissecting endoscope 10 is removed from the body (if it was used to perform the dissection of the pericardium at a location posterior to the inferior vena cava, otherwise dissecting endoscope may already be out of the body) and tip 20 is removed and replaced by tip 20′.

Endoscope 10 is then reinserted into the body and manipulated (under direct visualization through endoscope 10) to direct tip 20′ though suture or wire loop 36. Suture loop 36 is then cinched around tip 20′ in a manner as described above, with the difference being that suture loop 36 is cinched down over tapered region 20p and to abut against the proximal portion of ball 20d as shown in FIG. 5C. Since the cinched-down suture loop 36 is distal of the distal end of the endoscope shaft 16d, the procedure may be continuously and uninterruptedly viewed via the endoscope. Thus, not only can the surgeon see to position tip 20′, but the surgeon may also view the relative positions of tip 20′, including ball 20d, and suture loop 36, as ball 20d passes into and through loop 36. Further, the surgeon may also visually confirm that the loop 36 cinches down on the tapered portion of tip 20′ and can then continue to view the connection between catheter 30 and dissecting endoscope 10 (i.e., tip 20′ captured by suture loop 36) as dissecting endoscope 10 and catheter 30 are pulled out of the body. The enlarged cross-section of ball 20d ensures that suture loop 36 does not slip off of tip 20′, as long as suture loop 36 remains adequately cinched down. Tip 20′ may also be provided with an inner tapered lens 24 like that shown in FIG. 1B.

Further alternatively, all functions of dissecting endoscope may be carried out with tip 20′ in place. In this case, dissecting endoscope may also be alternatively provided with a fixed, non-removable tip in the configuration of tip 20′. In either of these situations, tip 20′ may be further provided with a small (e.g., about 1 mm diameter) nipple or protrusion 22 to extend from the distal end of tip 20′, as shown in FIG. 5B, or knurled spot, to increase friction with the tip 20′ against tissue to facilitate dissection.

FIGS. 6A-6E illustrate an alternative configuration the may be used to lock catheter 30 to endoscope 10. In this arrangement, tip 20″ is provided with a relatively inflexible or rigid loop 21 and a second, relatively inflexible or rigid loop 36′ extends distally from the distal end of catheter 30, as shown in FIG. 6A. Loops 21 and 36′ may be made of stainless steel wire, rigid polymer, or other substantially inflexible, biocompatible material. Loops 21 and 36′ are configured to be oblong or elliptical, and loop 21 is dimensioned to be inserted through loop 36 when rotated appropriately (FIG. 6B) by maneuvering endoscope 10. As loop 21 is inserted through loop 36′, device 10 is then maneuvered to rotate loop 21 back to its original orientation (FIG. 6A) with respect to loop 36′ (see FIGS. 6C and 6D), thereby locking endoscope 10 and catheter 30 together as shown in FIG. 6E, as loops 21 and 36′ function to effectively clasp device 10 and catheter 30 together.

As loop 21 is distal to the end of the endoscope shaft 16, both loops 21,36′ are visible through the endoscope (dissecting endoscope 10) at all times during locking together of the loops as well as during pulling the endoscope 10 and catheter 30 out of the body. While tip 20″ is shown to have a conical taper, it is not limited to this shape but may be essentially any other blunt shape, e.g., shaped like tip 20 or some other blunt shape. Tip 20″ may be permanently fixed to dissecting endoscope 10 or may be removable for replacement by a different tip.

Snare catheter 30 may be about 6 French in diameter, as noted previously, and may be about 60 to about 70 cm in length. The distal tip of catheter 30 may have a rounded end or may be connectable with a separately attachable tip 31, see FIG. 7A. Tip 31 is rounded may be formed of rigid, biocompatible polymer (e.g., polycarbonate, nylon, etc.) or metal (e.g., stainless steel). Tip 31 may be bonded (e.g., glued or adhered) to the catheter, where the rigidity it provides ensures that the ball tip is securely captured by the snare so that the snare does not slip off during traction. Initially, the snare (i.e., suture loop 36) may be provided to have a size of about 3-5 cm in diameter 36d, although this is of course variable and can be set according to the surgeon's preference. Suture line 34 may be constructed of one or more strands of suture 34s that is knotted 34k at one end. Additionally, the one or more suture or wire strands may be encased in a plastic, heat shrink tubing 34t to form an elongated body that runs the length of catheter 30 (within catheter 30). Note, elongated body 34s,34t is shown removed from catheter 30 in the exploded view of FIG. 7A).

Alternatively, suture or wire loop 36 may be glued or otherwise fixed inside a small plastic tube that forms the elongated body/suture line 34 that runs through catheter and extends proximally therefrom. In any case, the body of suture loop 36, as well as suture line/elongated body 34 have sufficient column strength to allow an operator to push on suture line 34 from the portion that extends proximally from the proximal end of catheter 30, to enlarge the size of loop 36.

Catheter 30 may be provided with one or more transverse holes 39 (see FIGS. 7A and 7B) extending through a proximal end portion thereof, sized to permit passage of sutures or other lines that may form distal leader 52, therethrough, to be tied to connect catheter 30 and ablation probe 50.

Another alternative procedure includes substituting a long tube 70 in place of the tube 14 and catheter 30 used as described in the previous procedures. FIG. 8A illustrates an example of such tube 70. Tube 70 may be spirally slit 72 along at least a portion of the length of tube 70 and typically is spirally slit 72 over the entire length of tube 70 as indicated in FIG. 8A. Further alternatively, tube 70 may be provided with only a single slit 74 that circumscribes a portion of the circumference of tube 70 that is long enough to form an opening to receive dissecting endoscope therethrough. Typically slit 74 is formed at a distance from the distal end of tube 70 that is about the same length as tube 14. In all of the alternative examples of tube 70 described, tube 70 retains sufficient column strength so that tube 70 can be advanced along a pathway around the pulmonary veins by pushing on the proximal portion of tube 70.

Additionally, suture loop 36 is positioned to extend from the distal end of tube 70 during use, as shown in FIG. 8C. Any of the variations of suture line 34 and loop 36 may be employed as described for previous examples. Further, tube 70 may have a small diameter catheter 76 mounted therethrough (see FIG. 8D), through which suture line 34 may be threaded. This may help to maintain suture line 34 and suture loop 36 in a static position relative to tube 70 while tube 70 is being advanced through the body. Tube 70 should at least be as long as to have the proximal end extending out of the patient when the distal end of tube 70 is in the oblique pericardial sinus, at least to the extent that suture loop is shown positioned in at FIG. 2B.

When using tube 70, the preliminary steps of forming the opening 11 in the patient's right chest wall, and dissecting, including dissecting pericardium to open an entrance to the transverse pericardial sinus 4, can be carried out by any of the techniques and devices/various combination of devices discussed above. However, instead of having tube 14 mounted over dissecting endoscope 10 during the dissection procedures, dissecting endoscope 10 is inserted through a portion of tube 70 prior to initiating the dissection procedures. Note also, that dissection endoscope 10 could be withdrawn back out of the patient after forming the opening to the transverse pericardial sinus, if preferred, to then insert the dissecting endoscope 10 through a portion of tube 70 and then reinsert dissection endoscope 10 along with tube 70 into the patient. However, this requires an extra step and accordingly, dissection is typically performed with tube 70 already in place over dissecting endoscope 10.

Using a spirally slit tube 70 offers the advantage that dissecting endoscope can be inserted through any of the various slits, thereby allowing the surgeon to tailor the length of the portion of tube 70 that will reside over the shaft of dissecting endoscope 10. However, a tube 70 with a single slit may be used in the same manner as described herein, with the difference being that the length of the portion of tubing 70 residing on the shaft of dissecting endoscope 10 will be predetermined. As another alternative, a tube having several slits 74 may be provided to give the surgeon some choice in the length of the portion of tubing 70 that will be slid over the shaft of dissecting endoscope 10. Whatever variation of tube 70 is used, suture loop 36 extends distally from the distal end of tube 70 after mounting on dissecting endoscope 10, as shown in FIG. 8C.

Upon entering transverse pericardial sinus 4 with the distal end of dissecting endoscope 10, tube 70 is advanced distally so that the distal end portion of tube 70 enters transverse pericardial sinus 4 and dissecting endoscope 10 is then removed from within tube 70 and removed from the body of the patient through opening 11. The slit 72,74 in tube 70 through which dissecting endoscope had been inserted closes upon removal of dissecting endoscope 10 and tube 70 assumes the shape of a continuous tube. Tube 70 is advanced inferiorly along the left border of the pericardium and into oblique pericardial sinus 7 (along the pathway described in the previously described methods) by pushing and manipulating tube 70 from its proximal end portion outside the body.

Any of the techniques or combinations of the same, for dissecting and creating an opening to the transverse pericardial sinus may then be used to provide the opening to the oblique pericardial sinus. Dissecting endoscope 10 may then be used to identify the distal end of tube 70 and suture loop 36,36′. Tip 20,20′,20″ may then be passed through loop 36,36′ respectively to join tube 70 to dissecting endoscope 10 in any of the ways described previously. The distal end of tube 70 is then manipulated to pull it out of opening 11 (either using single opening 11 procedures or dual-opening 11,13 procedures, both of which are described in detail above).

Next, ablation probe 50 may be inserted through the opening at the proximal end of tube 70 and advanced until it exits the distal end of tube 70, with tube 70 acting as a low friction guide for placement of ablation probe 50. Tube 70 is then removed from the patient's body by pulling on one end of the tube (typically the distal end) while holding the proximal end of ablation device 50 to make sure that it is not displaced from its proper orientation around the pulmonary veins. Endoscopic graspers may next be used to grasp the distal portion of ablation probe 50 to insert it back into the mediastinum. Epicardial ablation probe 50 is positioned to completely encircle the four pulmonary veins, and may be held in position using graspers 62 while energy is being delivered to accomplish the epicardial ablation. After completing the ablation, ablation device 50 is then pulled out of the chest and follow-up steps are carried out to close the opening in the patient and complete the surgery.

Further alternatively, tube 70 may be used in procedures using two openings 11,13, similar to those procedures described above that use two openings, only with the use of tube 70 instead of a snare catheter 30 and tube 14. Ablation probe 50 may be advanced manually through tube 70.

Further alternatively, when using operating endoscope 60 with endoscopic graspers 62, tube 70 need not have a suture loop, as graspers 62 may be used to grasp tube 70 and pull it out of opening 11. Alternatively, dissecting endoscope may be inserted into opening 11 and maneuvered to again pass tip 20 through suture loop 36, 36′, after which suture loop 36,36′ can again be cinched down/clasped in the same manner as described earlier. Once so connected, tube 70 can then be drawn out of opening 11 by pulling endoscope 10 back out of opening, after which endoscope may again be disconnected from tube 70 in the same way as discussed previously. Either technique results in both ends of tube 70 protruding out of opening 11.

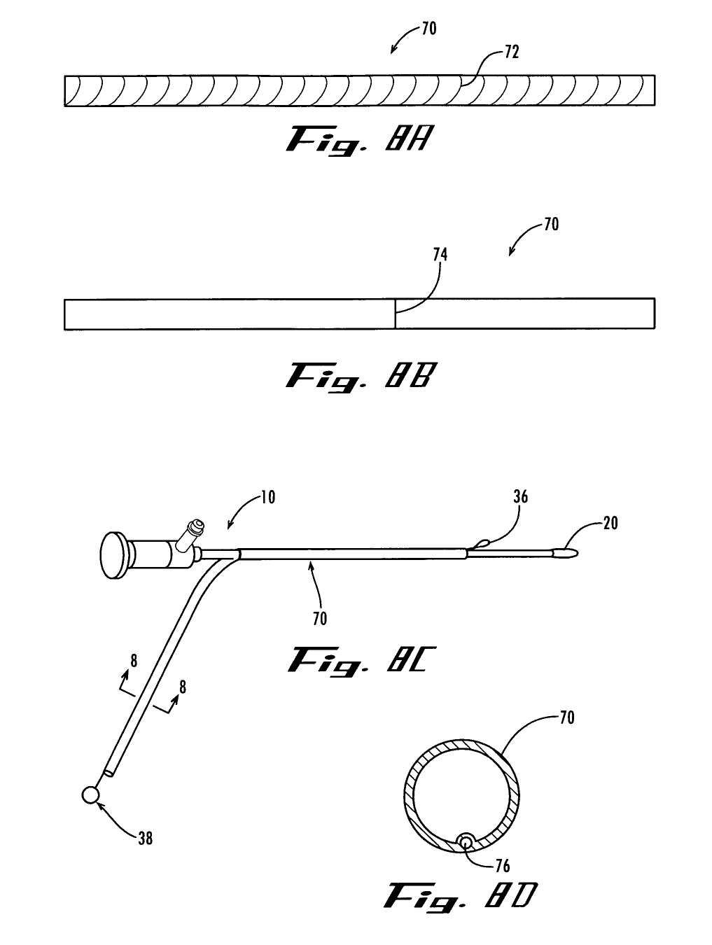

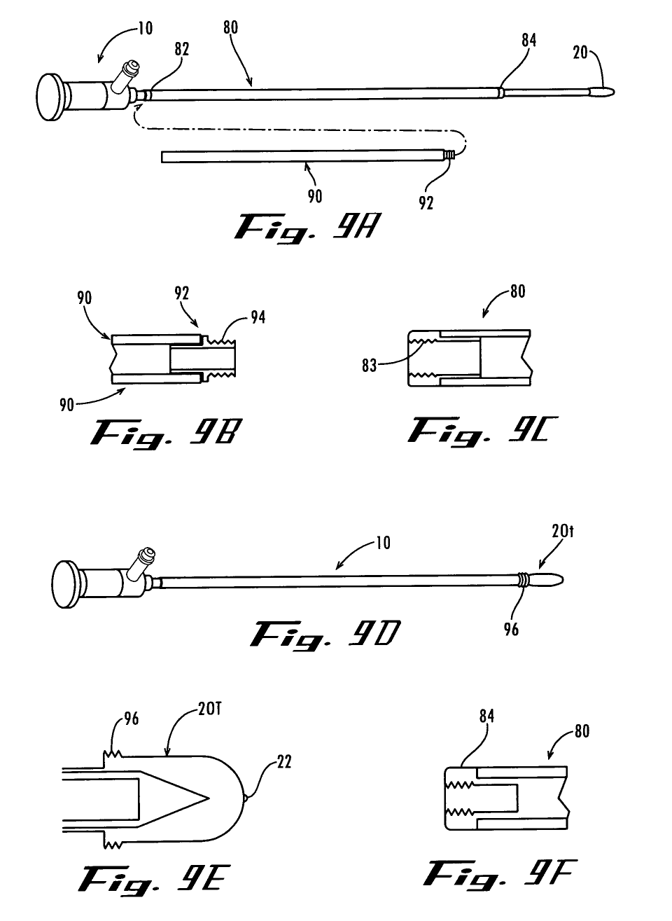

Further alternatively, two separate lengths of tubing 80 and 90 may be used to position an ablation probe into a desired position for performing an ablation. For example, tube 80 may be similar in length and material to tube 14 discussed above. Additionally, the ends of tube 80 include connectors 82 and 84, respectively, that are configured to connect with connectors on other components as described hereafter. Initially, tube 80 is mounted over dissecting endoscope 10 in the same manner as described with regard to tube 14 above. Tube 80 may be about two-thirds the length of shaft 16 for example. After endoscope 10 has completed dissecting posterior to the superior vena cava and tip 20 lies in the transverse pericardial sinus, tube 80 is advanced distally into the transverse sinus. Endoscope 10 is then removed from inside tube 80 and the patient's body, and tube 90 is next connected to tube 80. Tube 90 includes a connector 92 at its distal end that is configured to mate and forms a connection with connector 82 at the proximal end of tube 80. In the example shown in FIG. 9A, connector 92 includes male threads 94 and female connector 82 includes female threads 83. Of course, the mating connectors 82 and 92 are not limited to this configuration, as the threads on each may be reversed so that connector 82 has male threads and connector 92 has female threads, for example, or various other connection mechanisms may be substituted, including, but not limited to bayonet connectors, detent balls and mating recesses, etc.

The resulting long, connected tube formed from tubes 80 and 90 is then distally advanced by pushing on/manipulating tube 90 to advance the distal end of tube 80 to track downward along the left border of the pericardium, lateral to the left pulmonary veins 5, and into the oblique pericardial sinus 7. Next, or in the meantime, tip 20,20′ will have been removed from the distal end of dissecting endoscope 10 and replaced with dissecting connector tip 20t (FIGS. 9D,9E). Endoscope 10 is then reinserted through opening 11 (or inserted through an inferior opening 13, in an alternative dual-opening procedure) and used to dissect posterior to the inferior vena cava, to enter the oblique pericardial sinus 7. Alternatively, this dissection may be performed prior to changing tips, after which dissecting endoscope 10 may be removed from the patient to change tips. In this alternative procedure, connector tip 20t may or may not be provided with a dissection nub 22 or other feature for increasing friction between tissue and tip 20t to facilitate dissection. Connector tip 20t may have any or all of the features described with regard to tip 20 or alternatively may be of another blunt shape, for example.

In any case, connector tip 22t is configured to mate with and form a connection with connector 84 at the distal end of tube 80. In the example shown in FIG. 9D and the enlarged view of FIG. 9E, connector tip 20t is a dissecting tip having a protrusion, nub or nipple 22, and further includes male threads 96 configured to mate with female threads provided in connector 84. Of course, the mating connectors 20t and 84 are not limited to this configuration, as other connection mechanisms may be substituted, as would be readily apparent to one of ordinary skill in the art. Connector 84 may be identified by viewing through dissecting endoscope 10, and maneuvering endoscope 10 to align connector tip 20t with connector 84 and then advancing connector tip 20t and rotating into connector 84 to join dissecting endoscope 10 with tube 80,90. Dissecting endoscope 10 is then removed through opening 11, and disconnected from connector 84, leaving tube 80,90 encircling the pulmonary veins with the proximal end of tube 90 and the distal end of tube 80 extending out of the body through opening 11. In a two-opening procedure, dissecting endoscope 10 is advanced through inferior opening 13 to connect with tube 80,90. After connection with connector 84, dissecting endoscope 10 is pulled out of inferior opening 13 and disconnected from connector 84. The distal end of tube 80 (including connector 84) is reinserted into the right pleural cavity, and dissecting endoscope 10 is inserted through superior opening 11 and used to identify connector 84 again and to reconnect to connector 84 via connector tip 20t. Dissection endoscope is removed through opening 11, thereby leaving tube 80,90 encircling the pulmonary veins, with both ends of tube 80,90 exiting the superior opening 11.

Next, ablation probe 50 is advanced through tube 90,80, by manually pushing probe 50 through tube 90,80, to position it in the desired orientation around the pulmonary veins. Tube 80,90 is then removed by pulling the distal end of tube through the opening 11, while ensuring that ablation probe 50 is not advanced with the advancement of tube 80, 90, by holding onto the probe 50 with one hand, for example, until tube 80,90 is completely removed from the body, leaving ablation probe 50 in place for performing the epicardial ablation.

Endoscopic graspers may next be used to grasp the distal portion of ablation probe 50 to insert it back into the mediastinum. Epicardial ablation probe 50 is positioned to completely encircle the four pulmonary veins, and may be held in position using graspers 62 while energy is being delivered to accomplish the epicardial ablation. After completing the ablation, ablation device 50 is then pulled out of the chest and follow-up steps are carried out to close the opening in the patient and complete the surgery.

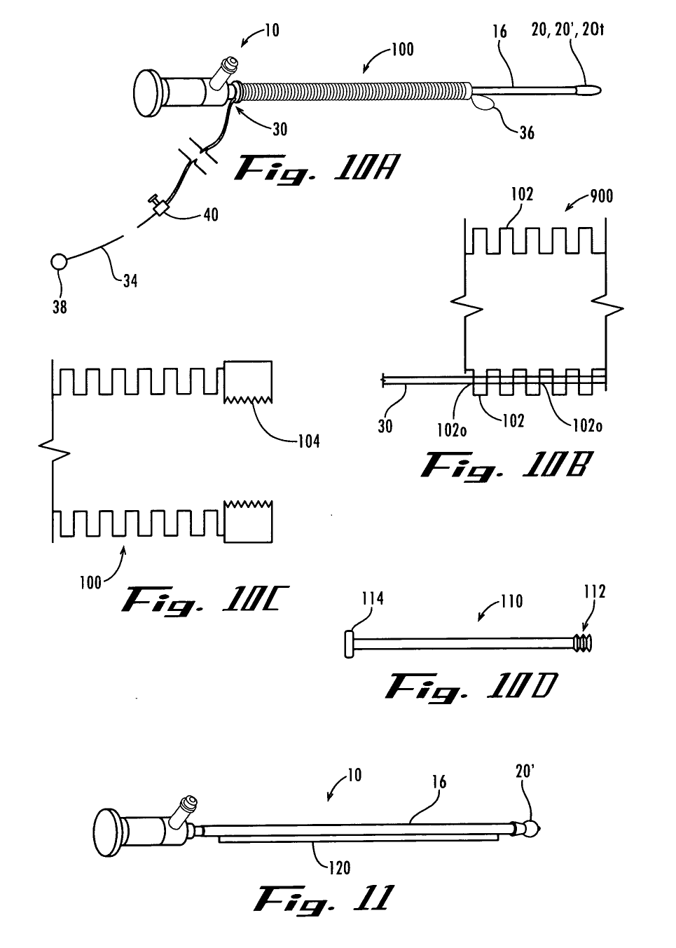

Another variation involves the use of expanding length tube 100 as illustrated in FIG. 10A. Tube 100 may be a corrugated tube constructed of a plastic material such as polyethylene, polyvinyl chloride, nylon, or polytetrafluoroethylene, for example. The corrugations permit tube 100 to be reduced in length by compression, and also allow tube 100 to bend without kinking. Tube 100 is shown in a compressed state in FIG. 10A. A small diameter lumen may reside on the inside of tube and run the length of tube 100 to allow snare catheter 30 to be threaded therethrough. Alternatively, snare catheter 30 may be passed though holes or other openings in the corrugations 102 of tube 100, provided outside the minor diameter of corrugated tube 100, as shown in the enlarged partial view of FIG. 10B, such that catheter 30 is freely slidable with respect to corrugations 102 and tube 100.

Suture loop 36 extends from the distal end of expandable tube 100 and from catheter 30 as described above, as suture line 34 runs through the inner smaller diameter lumen or through catheter 30, depending upon the particular variation used, and may be connected at a distal end to pull tab 38. Further, a lock 40 may be provided, as described in earlier embodiments. In a relaxed or uncompressed state, elongatable/expanding length tube 100 may be about twice the length of shaft 16 and tip 20 combined. When fully compressed, tube 100 may be about two-thirds the length of shaft 16, as shown in FIG. 10A. Tube 100 includes a connector 104 at a distal end thereof that is configured to mate with a connector 112 on mounting tube 110 to form a connection therewith. As shown in FIG. 10C, connector 104 includes female threads dimensioned to mate with male threads on connector 112 (FIG. 10D), although various other connection mechanisms may be substituted. Expandable tube 100 is compressed as it is mounted over mounting tube 110. Mounting tube 110 includes a flange 114 at its proximal end to act as a stop against the proximal end of tube 100 so that it can be compressed for mounting. When fully compressed, tube 100 may then be rotated with respect to tube 110 to screw connector 104 onto connector 112, thereby locking tube 100 in the compressed configuration. Tube 110 may be mounted over device 10 either prior to or after compressing and mounting tube 100 on tube 110.

After performing the dissection into the transverse pericardial sinus 4, such as by using dissecting endoscope 10 in a manner as described above, endoscope 10 and tube 110 are held steady, such as by preventing flange 114 from rotating, for example, and tube 100 is rotated to unscrew connector 104 from connector 112, thereby freeing tube 100 to expand back toward the relaxed, uncompressed configuration. As it is released, the operator may rotate tube 100, which may help to maneuver tube 100 through the transverse sinus, as it expands. Once expandable tube 100 has expanded over the distal end of dissecting endoscope 10, dissecting endoscope 10 may be removed from the body, and then tube 100 is further manipulated to advance it through the desired pathway, tracking down along the left border of the pericardium and into the oblique pericardial sinus. Even in its relaxed, uncompressed state, tube 100 has sufficient column strength to allow it to be pushed from a proximal portion thereof to advance the distal end of tube 100, in spite of the presence of some friction along the tissue surfaces against tube 100. This arrangement allows a long tube 100 to be compressed and loaded on dissecting endoscope 10 while exposing the distal third of endoscope 10 and thereby providing a small profile endoscope for dissection posterior to the vena cavae.

Once expanded and manipulated into the oblique pericardial sinus, the procedure may be further carried out in any of the manners discussed above with regard to elongated tube 80,90. A connection between tube 100 and dissecting catheter 10, to pull tube 100 from the oblique pericardial sinus out of the body, may be made using suture loop 36 for a snare-type connection, in any of the manners previously described, or using connector tip 20t to mate with connector 104, for example.

FIG. 11 shows another variation of a dissection endoscope designed to reduce the number of steps required to place ablation probe 50 in that no separate tube is required to be placed and then removed. In this variation, a small lumen or channel 120 runs parallel to the scope shaft 16 and has an inside diameter sufficient to push snare catheter 30 through. Using this device, once the dissection into the transverse pericardial sinus 4, such as by using this device in a manner as described above with regard to other variations of dissecting endoscope 10, and at least the tip 20,20′ and distal end of channel 120 have been inserted into the transverse pericardial sinus 4, snare catheter 30 may then be pushed through channel 120 until suture loop 36 emerges distally of the distal end of channel 120. Continued advancement of catheter 30 may be performed, while being viewed through endoscope 10 until loop 36 and the distal end of catheter reach the pericardium 2 on the left side of the heart and are deflected downwardly, and the distal end of catheter 30 and suture loop 36 are then further advanced into the oblique pericardial sinus 7, such as described above, and then dissecting endoscope 10 may be removed from the transverse sinus 4. Alternatively, dissecting endoscope 10 may be removed from the transverse sinus 4 once the distal end of catheter has been advanced out of the distal end of channel 120 and then catheter 30 can be further advanced into the oblique pericardial sinus.

In either case, dissecting endoscope may then be reinserted through opening 11, or inserted through opening 13, depending upon whether a single opening or dual opening procedure is being performed, to approach suture loop 36 to be snared thereby in a manner as described above. The remainder of these procedures may be carried out in the same manner as described in previous examples that used a snare catheter 30 to draw ablation probe 50 into place.

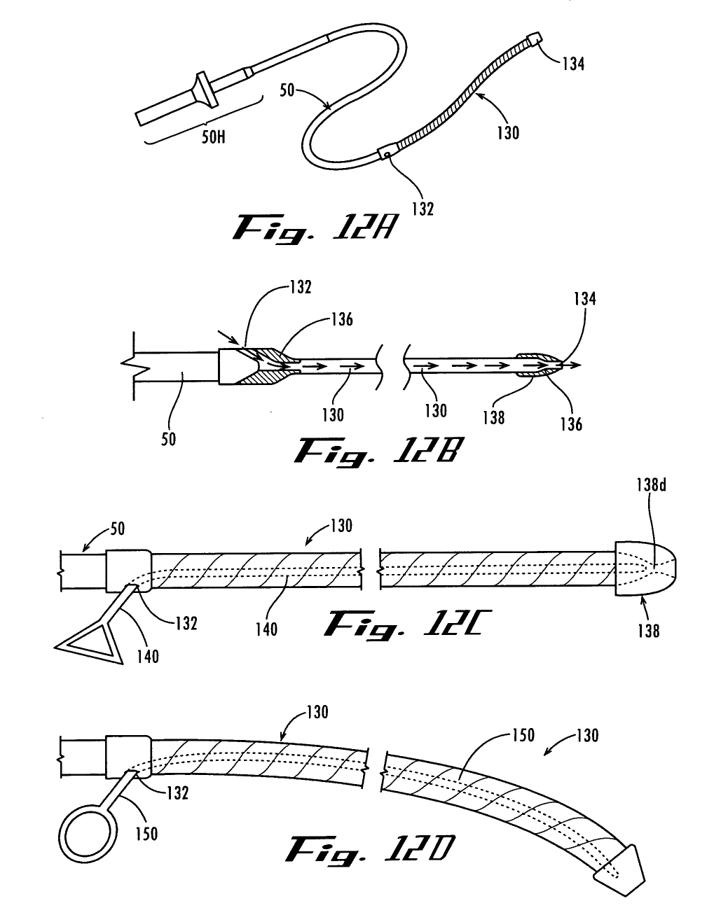

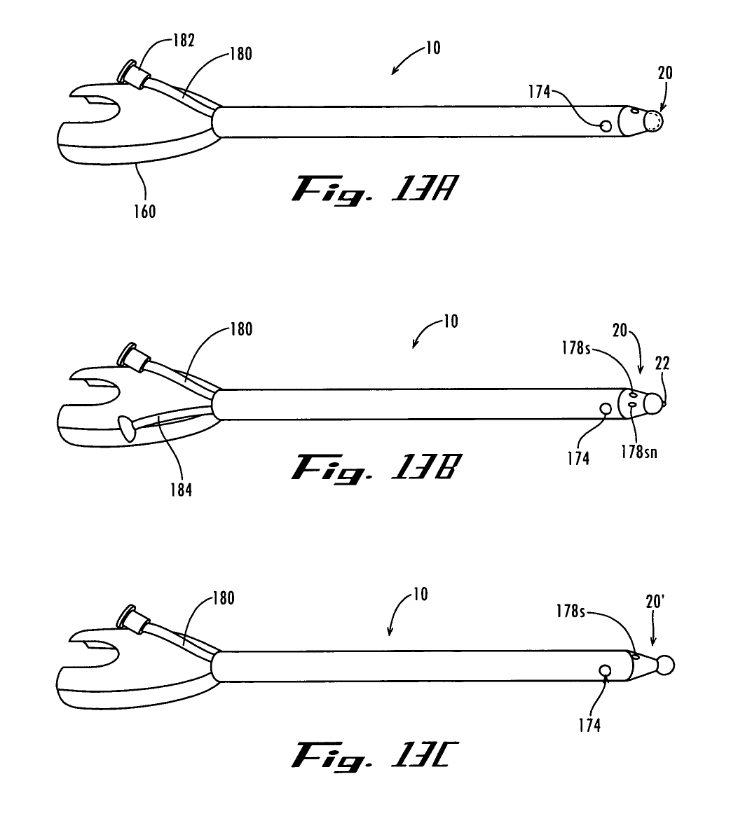

Another approach to reducing the number of steps required to place ablation probe 50 around the pulmonary veins 5,6 is exemplified in FIGS. 12A-12B. This approach includes connection of torque tube 130 to the distal end of ablation probe 50. Note that ablation probe 50 may be provided with a proximal handle 50H. Torque tube 130 and ablation probe 50 may be integrally formed or torque tube 130 may be welded to an end cap on probe 50. Further alternatively, the end cap on probe 50 may have a distal hole that is internally threaded and the proximal end of torque tube 130 may have mating external threads so that the proximal end of torque tube 130 can be screwed into the end cap of probe 50. Alternatively or additionally, the two components 50,130 may be glued, welded or encased in a plastic sleeve to combine them. Torque tube 130 is flexible in bending but rigid with respect to torsion about the longitudinal axis. Torsion tube may be a “spring tube”, e.g., a coil spring covered by a sleeve of silicone rubber or plastic sheathing (polyurethane, polyvinylchloride, polyethylene, or the like), and replaces the need for a separate routing tube such as tube 14, tube 70 or tubes 80,90, for example.

In addition to guiding ablation probe along the desired pathway around the pulmonary veins, torque tube 130, by its torsional rigidity property, is configured to also facilitate the torsional control of the ablation probe 50. Ablation probe 50 has a particular side that is designed to be placed against the tissue to be ablated, to optimally space the ablation element (antennae) at the desired distance from the tissue for delivery of energy thereto to perform the ablation (in the case of a microwave probe; alternatively, the intended side for contact with the tissue may place an ablation element into direct contact with the tissue, e.g., as in the case of use of an Rf ablation element). If such particular side is not in contact with the cardiac tissue when ablation probe 50 has been pulled into position to surround the pulmonary veins, then torque tube 130 can be rotated about its longitudinal axis to deliver torque to ablation probe 50 so as to rotate the probe 50, until the appropriate side of the probe is in contact with the tissue.

At the proximal end of torque tube 130, an opening or entry hole 132 may be provided, to allow another instrument to be inserted into the torque tube 130. Torque tube 130 is annular and therefore open space is provided between opening 132 and a distal opening 134 in torque tube, as shown more clearly in the sectional view of FIG. 12B. Opening 132 and well as distal opening 134 may be surrounded by smooth, ramped/funneled inner walls 136 to provide for a smooth entry and exit of an instrument introduced therethrough. Thus, for example, a stylet, or snare catheter 30 may be inserted into torque tube 130, as will be described in more detail below.

In using the arrangement shown in FIGS. 12A-12B, after dissecting the pericardial reflection posterior to the superior vena cava as described previously, torque tube 130 may next be inserted into the transverse pericardial sinus 4 with the aid of a straight stylet 140 inserted into torque tube 130 (FIG. 12C) to provide it with some rigidity and make it easier to control during the insertion. The cross-sectional diameter of stylet 140 is greater than the smallest inside diameter within nose cone 138, so that stylet 140 is prevented from extending out of distal opening 134. Further, stylet 140 may be pulled out of torque tube 130 at any time or any stage of the procedure as desired, to allow torque tube to take its own course as it is advanced toward the pericardium 2 on the left side of the heart and deflected downwardly toward the oblique pericardial sinus 7, as this may cause less trauma to the surrounding tissues that when torque tube is directed along a straight path by stylet 140.

In order to direct the distal end of torque tube 130 downwardly, a slightly curved stylet 150 may be inserted into torque tube 130 as shown in FIG. 12D. Additionally or alternatively, a malleable stylet may be provided so that the curve can be adjusted as desired by the operator. As with the use of stylet 140, the cross sectional diameter of curved stylet is larger than inside diameter 138d so that the curved stylet will never protrude out of the distal end of torque tube 130. Curved stylet may be inserted after use of straight stylet 140, as described above has been used to deliver the distal end of torque tube 130 to the pericardium 2 on the left side of the heart. By removing straight stylet 140 and inserting curved stylet 150, the nose cone 138/distal end of torque tube 130 is directed downwardly, and may be also directed somewhat posteriorly toward the direction of the oblique pericardial sinus. Further advancement of torque tube 130, after removal of stylet 150 once the distal end of torque tube has been properly pointed or directed, delivers the distal end of torque tube 130 into the oblique pericardial sinus 7. Alternatively, curved stylet 150 may be used in the same manner as described above, but after advancement of torque tube without use of a straight stylet 140, or where partial advancement with a straight stylet 140 was carried out, with partial advancement using no stylet. Still further, curved stylet may be used for the entire insertion, beginning from entering into the transverse pericardial sinus 4, up until the point where the distal end portion of torque tube 130 is pointed toward the oblique pericardial sinus 7.

Once the distal end of torque tube 130 is directed toward the oblique pericardial sinus 7, and stylet 150 has been removed from torque tube 130, further advancement of torque tube 130, by pushing from a location outside of the body, drives the distal end toward the oblique pericardial sinus 7, as the pericardial sac on the left hand side guides torque tube 130 toward the desired location. Once the distal end of torque tube 130 has been delivered to a desired location in the oblique pericardial sinus, snare catheter 30 may then be inserted through entry hole 132 and advanced through torque tube 130 until suture loop 136 extends distally from opening 134. At this stage, any of the procedures for inserting dissecting endoscope 10 into oblique pericardial sinus 7 and snaring a tip of the endoscope 10 with suture loop 136 may be carried out to connect torque tube 130 to dissecting endoscope 10. Note that the proximal portion line that extends proximally out of opening 132 needs to be locked, such as by using lock 40, for example, to prevent additional suture line 34 from advancing into catheter 30 as torque tube is being pulled out of the body by pulling on dissecting endoscope.

Additionally, snare catheter 30 needs to be locked with respect to torque tube 130, so that is does not advance further into torque tube 130 as torque tube 130 is being pulled by dissecting endoscope 10. Still further, snare catheter 30 should be sufficiently long so that the locked proximal portions of catheter 30 and suture line 34 still extend from opening 11 even when the distal end of torque tube 130 is pulled out of the patient. This makes it easier to unlock the locks, and increase the size of loop 36 to release the dissection catheter. After such release, snare catheter may be pulled out of torque tube 130 and the body by pulling from the proximal portion of snare catheter that extends out of the body.

The connection between torque tube 130 and ablation probe 50 makes it unnecessary to tie leads 52 through openings, thereby eliminating that step required when using alternative procedures. In fact the leads 52 can be eliminated altogether. Torque tube 130 may be disconnected in some situations before the distal end of ablation probe 50 is placed back into the mediastinum, to make it easier to manipulate and encircle the pulmonary veins. However, such is not necessary. As in earlier described procedures, the snare catheter is pulled out of the body using the endoscope tip 20,22, bringing the ablation probe 50/torque tube 130 with it. The snare catheter is removed and torque tube 130 may be removed before ablation probe is returned to the mediastinum, as already noted. An endoscopic grasper may be used to position probe 50 around the pulmonary veins to perform ablation.

Another alternative procedure involves the use of dissecting endoscope 10 to dissect the pericardial reflection underneath the superior vena cava, using tip 20. Next dissecting endoscope 10 is pulled back out of opening 11 and tip 20 is removed and replaced by tip 20′. Loop 36 is cinched down over tip 20′ to connect snare catheter 30 to dissecting endoscope 10 and together, dissecting endoscope 10 and snare catheter 30 are inserted into the transverse pericardial sinus and the distal end of snare catheter may be advanced by dissecting endoscope 10 over to reach the pericardium 2 on the left side of the heart, where loop may then be loosened and dissecting catheter 10 may be removed for further manipulation of catheter 10 to deflect the distal end downwardly for advancement into the oblique pericardial sinus. All further steps and techniques for this procedure have been previously described above with regard to alternative procedures using snare catheter 30.

Alternative to advancing the distal end of snare catheter 30 over to pericardium 2 on the left side of the heart, dissecting endoscope may be released and removed after initially positioning the distal end of catheter 30 through the opening in the pericardium and into transverse pericardial sinus 4, after which, catheter 30 can be advanced to find its own pathway over to the pericardium 2 on the left side of the heart. Further alternatively, a tip 20′ with nub 22 may be used to perform the dissection of the pericardial reflection under the superior vena cava while suture loop 36 has already been cinched down to connect snare catheter 30 thereto. In this case, dissecting endoscope 10 does not need to be pulled out of the body after performing the initial dissection, but can instead be inserted directly into the transverse pericardial sinus with the snare catheter 30 already attached.

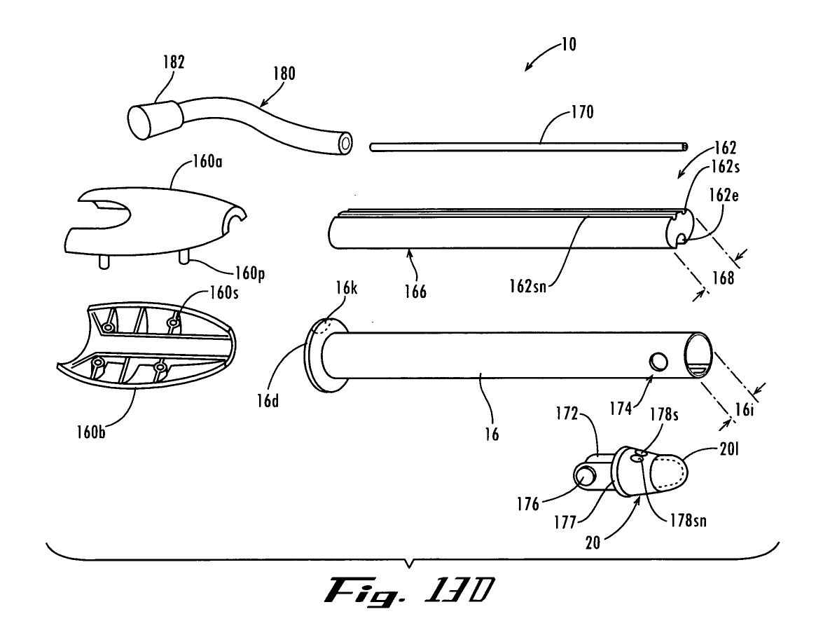

FIG. 13A shows another example of an instrument 10 useful for performing procedures for epicardial atrial ablation. Instrument 10 includes main tube 16 that is configured to receive an endoscope therethrough, similar to the instrument shown in FIGS. 1A and 1B. Additionally, other lumens may be provided within main tube 16 to permit greater functionality of device 10, as described in more detail below with reference to FIG. 13D.

FIG. 13D is an exploded representation of the device 10 shown in FIG. 13A. Main tube 16 is substantially rigid and may be made from stainless steel, or other biocompatible metal, alloy, rigid polymer or composite. The proximal end portion of main tube 16 is captured by handle 160. The proximal end of handle 160 is open to receive an endoscope that is guide therethrough and through main tube 16 via endoscope lumen 162 for viewing through tip 20. Handle 160 is typically formed in halves 160a, 160b that may be assembled over the proximal end portion of main tube 16, thereby capturing tube 16 to prevent axial movements with respect to handle 16. Tube 16 may be mounted to allow rotation with respect to handle 160 (as shown in FIG. 13D), or may be mounted to prevent rotation. In order to prevent rotation, for example, proximal disk or washer may be formed with one or more scallops 16k (shown in phantom in FIG. 13D) and handle 160 may then be provided with a mating projection or key that mates with scallop 16k thereby preventing relative rotation between handle 160 and tube 16 once handle 160 has been assembled on tube 16.

Handle 160 is rigid and may be made of any of the materials described above for making tube 16. Typically handle 16 is molded form a rigid polymer, such as polycarbonate, for example. Pegs 160p protrude from one portion 160a of handle and are provided to mate with sockets 160s provided in corresponding locations of the other portion 160b of handle 160. Handle 160 may be further secured upon assembly by screws, bolts, adhesives, or the like or combinations of the same.

Insert 166 is provided as a convenient way to form multiple lumens within main tube 16. Insert 166 has a major cross-section dimension 168 that is slightly less, but nearly equal to the inside diameter 16i of main tube 16, so that when insert 166 is inserted into main tube 16, it forms a friction fit with main tube 16. Alternatively, insert 166 may be configured to loosely slide within main tube 16, and upon insertion to the desired position, may be secured by one or more set screws or other mechanical and/or chemical expedient. Insert 166 is further provided with one or more grooves or “half-lumens” 162 that, together with the inside wall of main tube 16 form full lumens when insert 166 is positioned within tube 16. In the example shown, a large half lumen 162e is provided to form a lumen in device 10 through which an endoscope will be passed, half lumen 162s is provided to form a lumen to receive suction tube 170, and half lumen 162sn is provided to form a lumen to pass a snare catheter through. A further advantage provided by insert 166 is that multiple, interchangeable inserts 166 may be provided to change the lumen configuration of device 10. For example, an additional insert may be provided to form four lumens 162 with tube 16. By removing the insert 166 shown in FIG. 13D from tube 16 and inserting the insert with four half lumens (not shown), device 10 would then be configured with four lumens. Device 10 also is provided with the capability of interchanging tips 20, and therefore a tip having an additional through hole could also be interchanged to accommodate the additional lumen. Of course, inserts 166 with less than the number of half lumens 162 shown in FIG. 13D may be provided and interchanged to configure device 10 to have less than three lumens.

Tip 20 may be configured to also be interchanged, as noted above. In the example shown, tip 20 has prongs 172 extending proximally therefrom, with pins, pegs or other protrusions 176 extending therefrom. Tip 20 may further be optionally provided with a gasket or other seal 177 to prevent fluid flow into tube 16 where tip 20 meets tube 16. Main tube 16 is provided with openings 174 configured to receive protrusions 176, thereby locking tip 20 to main tube 16. Upon inserting, prongs 172 are flexed inwardly to allow protrusions 176 to pass within tube 16. The potential energy stored in prongs 172 by such flexing, drives prongs into openings 174 as the potential energy is converted to kinetic energy, and maintains them there, thereby locking tip 20 with respect to tube 16. To remove tip 20 for interchange, protrusions 176 are pressed inwardly to clear the walls of openings 174 and the tip can then be simply pulled out from its attachment with tube 16.

In the example shown, tip 20 is configured to provide the endoscope with an improved depth of field. The lens 201 of tip 20 is provided with a constant wall thickness throughout (and may be formed of clear polycarbonate, for example), and with a radius of curvature that allows the distal end of an endoscope to butt up against the inner surface of lens 201 and still be able to focus on tissues outside of the tip. As noted, tips may be interchanged to provide specialized functions. For example, device 10 in FIG. 13B shows the tip 20 of FIG. 13A having been interchanged with tip 20 having a protrusion 22, similar to tips having been described above to facilitate dissection. FIG. 13C shows device 10 in which tip 20 has been replaced by ball tip 20′.

Tip 20,20′ may be further provided with openings 178 that communicate with lumens 162 in device 10. For example, while endoscope lumen 162e directs to lens 201 of tip 20 shown in FIG. 13D, opening 178s fluidly communicates with lumen 162s so that suction can be delivered outside of tip 20. Additionally, opening 178sn communicates with lumen 162sn, permitting a snare catheter to be passed distally of device 10 through lumen 162sn and opening 178sn. In instances where more or fewer lumens are provided in device 10, as described above, more or fewer openings 178 may be provided in tip 20, respectively.

Suction tube 170 fluidly connects with suction luer 180, which may be made from TYGON® tubing or other vinyl, PAC or nylon surgical tubing. Suction luer 180 is further provided at a proximal end thereof with luer connector 182 configured to be connected with a source of vacuum, to thereby deliver suction to the distal end of device 10 through suction tube 170 and suction opening 178s. Similarly, an introducer tube 184 may be provided to connect with snare luer 162sn to guide a snare catheter into device 10, through snare luer 162sn and distally out of snare opening 178sn.

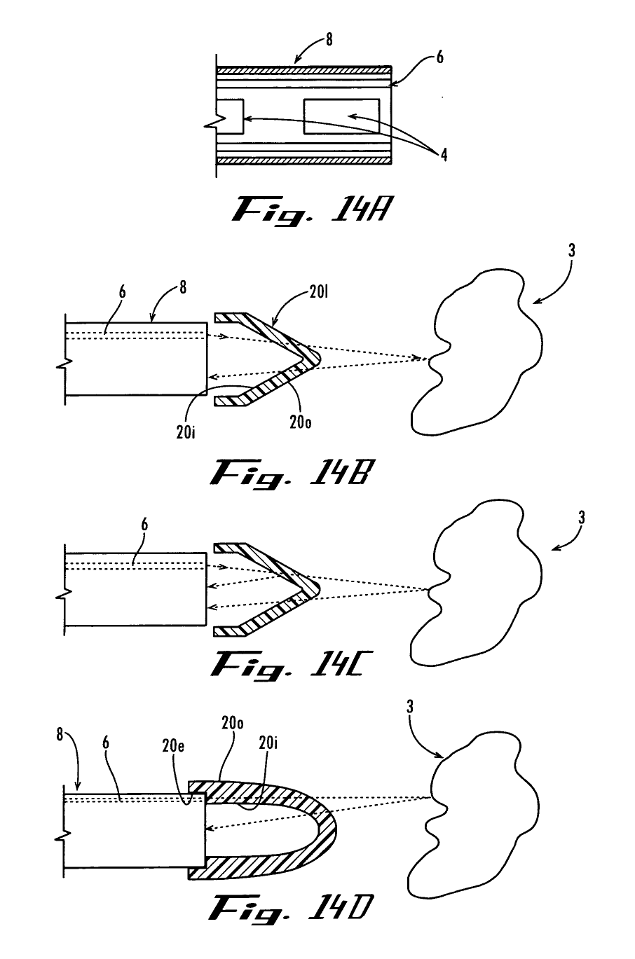

Turning now to FIGS. 14A-C, a discussion of an improved light delivery configuration for prevention of reflections from the lens of tip 20 to an endoscope viewing therethrough is discussed. FIG. 14A is a partial sectional, schematic drawing of an endoscope 8 showing a typical arrangement of one or more fiber optical light emitters 6 relative to lenses 4 that are used to view an image through the endoscope. With this typical arrangement, when endoscope 8 is combined in a device 10 having a tip 20 for viewing through the lens 201 of tip 20, fiber optic 6 typically is aligned to shine through the lens 201 as schematically represented in FIG. 14B.

As light is directed from the fiber optic 6 in the configuration of FIG. 14B it is made incident upon the inner wall 20i of lens 201 with the intention of passing the light beam through lens 201, and outside wall 20o, to impinge upon an object to be viewed through endoscope 8. Some of the light is then reflected from the object 3 and passes back through lens 201 and into the endoscope 8, through lenses 4 back to a viewing lens and/or camera for viewing an image of the object 3. However, in addition to the desired pathway of the light just described, part of the light from fiber optic that is made incident upon the inner surface 20i of lens 201 is reflected back to the endoscope 8 and is processed by the endoscope 8 as a bright ring or other shape of reflection that interferes with visualization of the object 3, as diagrammed in FIG. 14C.

In order to eliminate such reflections tip 20 may be designed with a lens 201 that interfaces with endoscope 8 such that fiber optic 6 is aligned with a proximal end surface 20e of lens 201. With this arrangement, light emitted from fiber optic 6 is made incident on the proximal surface 20e of lens 201 and directed between the walls 20i, 20o, and then exits outer wall 20o as it is directed onto the object to be viewed. With this arrangement there can be no light reflected from the inner surface of the lens 201 and this eliminates the problem discussed above. Since the proximal surface 20e is substantially parallel with the emission end of fiber optic 6, there is essentially no reflection of incident light back from surface 20e, but even if there is a slight reflection, the reflection is at so small an angle that it does not detrimentally effect the light viewed through lenses 4 since the angle is not great enough to direct such reflected light into lenses 4.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. An endoscopic procedure requiring access through only one side of a patient's chest, said procedure comprising the steps of:
advancing an instrument through an opening in the right chest of the patient;
dissecting the patient's pericardium to provide access to a transverse pericardial sinus;
advancing a lead through the pericardium and the transverse pericardial sinus and into an oblique pericardial sinus of the patient;
dissecting the patient's pericardium to provide access to the oblique pericardial sinus;
inserting an instrument into the oblique pericardial sinus; and
connecting the lead and the instrument together in the oblique pericardial sinus.

2. The procedure of claim 1, further comprising pulling the instrument out of the body, thereby drawing at least a portion of the lead out of the body.

3. The procedure of claim 1, further comprising connecting an ablation probe to a proximal portion of the lead.

4. The procedure of claim 3, further comprising pulling the instrument out of the body, thereby drawing at least a portion of the lead out of the body, and drawing at least a portion of the ablation probe into the body.

5. The procedure of claim 4, comprising drawing the lead out of the body to an extent sufficient to disconnect the ablation probe from the lead, disconnecting the ablation probe from the lead, and reinserting an end portion of the ablation probe into the chest of the patient so as to completely encircle the pulmonary veins with the ablation probe.

6. The procedure of claim 5, further comprising ablating cardiac tissue to form a lesion around the pulmonary veins.

7. The procedure of claim 2, wherein said advancing an instrument and said pulling an instrument out of the body are both carried out through a single opening and wherein the single opening is the only opening required to perform the procedure.

8. The procedure of claim 2, wherein said advancing an instrument is carried out through a first, superior opening and said pulling an instrument out of the body is carried out through a second, inferior opening.

9. The procedure of claim 8, further comprising disconnecting the instrument from the lead, reinserting the lead into the body through the second, inferior opening; advancing an instrument through the first superior opening, attaching the instrument to the lead, and drawing the instrument out of the first superior opening thereby drawing at least a portion of the lead out of the body.

10. The procedure of claim 9, including attaching an ablation probe to a proximal end portion of the lead, wherein said drawing at least a portion of the lead out of the body draws at least a portion of the ablation probe into the body to follow a pathway around the pulmonary veins established by the lead.

11. The procedure of claim 10, further comprising pulling the lead out through the first superior opening to an extent sufficient to disconnect the ablation probe from the lead, disconnecting the ablation probe from the lead, and reinserting an end portion of the ablation probe into the chest of the patient so as to completely encircle the pulmonary veins with the ablation probe.

12. The procedure of claim 11, further comprising activating the ablation probe to ablate cardiac tissue to form a lesion around the pulmonary veins.

13. The procedure of claim 4, wherein said advancing an instrument is carried out through a first, superior opening and said pulling an instrument out of the body is carried out through a second, inferior opening, said procedure further comprising disconnecting the instrument from the lead, reinserting the lead into the body through the second, inferior opening; advancing an instrument through the first superior opening, attaching the instrument to the lead, and drawing the instrument out of the first superior opening thereby drawing at least a portion of the lead out of the body.

14. The procedure of claim 13, said procedure further comprising drawing the lead out of the first superior opening to an extent sufficient to disconnect the ablation probe from the lead, disconnecting the ablation probe from the lead, and reinserting an end portion of the ablation probe into the chest of the patient so as to completely encircle the pulmonary veins with the ablation probe.

15. The procedure of claim 14, further comprising activating the ablation probe to ablate cardiac tissue to form a lesion around the pulmonary veins.

16. The procedure of claim 1, further comprising advancing a tube over the instrument and through the access to the transverse pericardial sinus, thereby cannulating an opening through the pericardium.

17. The procedure of claim 16, further comprising removing the instrument from the tube, wherein the lead is advanced through the tube, after said removing the instrument, during said advancing through the pericardium and transverse pericardial sinus.

18. The procedure of claim 1, wherein said lead comprises a snare catheter, said procedure further comprising passing a distal tip of said instrument through a loop extending from a distal portion of said snare catheter.

19. The procedure of claim 19, wherein said connecting comprises cinching said loop down around a distal end portion of said instrument.

20. The procedure of claim 1, wherein said instrument advanced through an opening comprises a dissecting endoscope.

21. The procedure of claim 1, wherein said dissecting is performed with a distal tip of said instrument having been advanced through the opening.

22. The procedure of claim 21, wherein said instrument having been advanced through the opening comprises a dissecting endoscope, said procedure further comprising viewing the dissection through the dissecting endoscope during said dissecting.

23. The procedure of claim 1, wherein the instrument inserted into the oblique pericardial sinus comprises an endoscope, said procedure further comprising viewing at least a distal portion of the lead through said endoscope while advancing the instrument toward the distal portion of the lead in the oblique pericardial sinus.

24. The procedure of claim 23, wherein the endoscope comprises a dissecting endoscope, said dissecting the patient's pericardium to provide access to the oblique pericardial sinus also being carried out by said dissecting endoscope.

25. The procedure of claim 24, further comprising viewing said dissecting the patient's pericardium to provide access to the oblique pericardial sinus, as said dissecting proceeds, through said dissecting endoscope.

26. The procedure of claim 18, wherein the instrument inserted into the oblique pericardial sinus comprises an endoscope, said procedure further comprising viewing at least said loop through said endoscope while advancing the endoscope toward the distal portion of the lead in the oblique pericardial sinus.

27. The procedure of claim 26, wherein said endoscope comprises a transparent distal tip, said procedure further comprising passing at least a portion of said distal tip through said loop, and said connecting comprises cinching said loop down around said endoscope.

28. The procedure of claim 27, wherein said tip comprises a ball tip and said loop is cinched proximally of a ball portion of said ball tip.

29. The procedure of claim 9, wherein said advancing an instrument through the first, superior opening and attaching the instrument to the lead comprises inserting graspers through the first superior opening and grasping a portion of said lead with said graspers.

30. The procedure of claim 29, wherein said instrument advanced through the first, superior opening further comprises an operating endoscope, said procedure further comprising viewing at least a portion of said lead through said operating endoscope during at least one of said advancing an instrument through the first, superior opening and attaching the instrument to the lead.

31. The procedure of claim 1, wherein said lead comprises a slit tube, said procedure further comprising inserting the instrument through a slit in said slit tube prior to said advancing the instrument through the opening in the right chest of the patient.

32. The procedure of claim 31, wherein a distal end portion of the instrument extends beyond a distal end of the slit tube after said inserting the instrument though a slit in said tube.

33. The procedure of claim 32, further comprising advancing the slit tube over the instrument and through the access to the transverse pericardial sinus, thereby cannulating an opening through the pericardium.

34. The procedure of claim 33, further comprising inserting an operating endoscope into the oblique sinus; advancing graspers via said operating endoscope; grasping a distal end of said slit tube; and retracting a distal end of said slit tube out of the body.

35. The procedure of claim 33, further comprising inserting the instrument into the oblique pericardial sinus; and advancing the slit tube concentrically over the instrument, using the instrument to guide the slit tube out of the body.

36. The procedure of claim 34, further comprising pulling the slit tube out of the patient, distal end first, leaving the ablation probe in position around the pulmonary veins.

37. The procedure of claim 16, wherein the tube is a first tube, said procedure further comprising removing the instrument from the first tube; connecting a distal end of a second tube to a proximal end of the first tube; and advancing the connected tubes into the body to move a distal end portion of the first tube into the oblique pericardial sinus, as tracks downward along the left border of the pericardium lateral to the left pulmonary veins.

38. The procedure of claim 37, wherein the instrument is connected to the distal end of the first tube via screw threads.

39. The procedure of claim 37, wherein the instrument inserted into the oblique pericardial sinus is a dissecting endoscope, said method further comprising viewing, through the dissecting endoscope, said connecting the lead and the instrument together as said connecting is performed.

40. The procedure of claim 37, further comprising pulling the instrument out of the body, thereby drawing a distal end portion of the first tube out of the body, wherein the instrument and distal end portion of the first tube are pulled out of a second opening in the right side of the patient that is inferior to the opening recited in claim 1, said opening recited in claim 1 comprising a superior opening in the right chest of the patient, said procedure further comprising disconnecting the instrument from the distal end of the first tube, and reinserting the distal end of the first tube through the inferior opening into the right pleural cavity.

41. The procedure of claim 40, further comprising inserting an instrument through the superior opening, connecting the instrument to the distal end portion of the first tube and pulling the instrument and distal end portion of the first tube out of the superior opening.

42. The procedure of claim 41, wherein the instrument advanced thorough the superior opening and connected to the distal end portion of the first tube comprises a dissecting endoscope, said procedure further comprising viewing the distal end portion of the tube, through the endoscope, and viewing said connecting the instrument to the distal end portion of the first tube.

43. The procedure of claim 41, further comprising advancing an ablation probe thorough said second and first tubes to a position to surround the pulmonary veins; and pulling the first and second tubes out of the body, leaving the ablation probe in position around the pulmonary veins.

44. The procedure of claim 43, wherein said pulling is in a direction to pull the first tube out prior to the second tube.

45. The procedure of claim 37, further comprising pulling the instrument out of the body, through the opening, thereby drawing a distal end portion of the first tube out of the body, through the opening,

46. The procedure of claim 45, further comprising advancing an ablation probe thorough said second and first tubes to a position to surround the pulmonary veins; and pulling the first and second tubes out of the body, leaving the ablation probe in position around the pulmonary veins.

47. The procedure of claim 46, wherein said pulling is in a direction to pull the first tube out prior to the second tube.

48. The procedure of claim 1, wherein the tube is a length expandable tube, and the length of the tube is increased by expanding the length expandable tube while passing the tube over the instrument, said procedure further comprising removing the instrument from with the tube when once the tube has advanced to the distal end of the instrument, wherein said advancing of the tube continues through the transverse pericardial sinus and into the oblique pericardial sinus.

49. A minimally invasive method of encircling the pulmonary veins of a patient, wherein said method requires entry into the patient from only one side of the chest, said method comprising the steps of:
advancing a lead through an opening in the chest of the patient, through a first opening in the pericardium, and into a transverse pericardial sinus of the patient, across the transverse pericardial sinus and into an oblique pericardial sinus of the patient as the lead tracks downward along a closed border of the pericardium on a side of the heart opposite to the opening in the pericardium;
inserting an instrument through a second opening in the pericardium and into the oblique pericardial sinus; and
connecting the lead and the instrument together in the oblique pericardial sinus.

50. The method of claim 49, further comprising pulling the instrument out of the body, thereby drawing at least a portion of the lead out of the pericardial sinus, through the second opening in the pericardium and out of the body.

51. The method of claim 49, wherein the opening is formed in the right chest of the patient, the lead tracks along the left border of the pericardium, lateral to the left pulmonary veins, and the second opening in the pericardium is inferior in position to the first opening formed in the pericardium.

52. The method of claim 49, further comprising advancing an instrument through the opening in the chest of the patient and through the first opening in the pericardium, prior to advancing the lead therethrough.

53. The method of claim 49, wherein the instrument inserted through the first opening comprises a dissecting endoscope.

54. The method of claim 53, further comprising inserting the instrument through at least a portion of the lead prior to said inserting the endoscope through the first opening.

55. The method of claim 53, further comprising dissecting the pericardium to form said first opening in the pericardium, prior to said advancing the instrument through the first opening in the pericardium.

56. The method of claim 54, further comprising dissecting the pericardium to form said first opening in the pericardium, prior to said advancing the instrument through the first opening in the pericardium.

57. The method of claim 49, wherein the instrument inserted through the second opening comprises a dissecting endoscope.

58. The method of claim 49, wherein the instrument inserted through the first opening is the same instrument inserted through the second opening, and wherein said instrument comprises a dissecting endoscope.

59. The method of claim 57, further comprising dissecting the pericardium with said dissecting endoscope to form said second opening in the pericardium, prior to said advancing the instrument through the second opening in the pericardium.

60. The method of claim 59, further comprising viewing at least a portion of said dissecting of the pericardium, through said dissecting endoscope.

61. The method of claim 57, further comprising viewing at least a portion of said step of connecting the lead and the instrument together, through said dissecting endoscope.

62. The method of claim 49, wherein said connecting comprises snaring the instrument with the lead.

63. Surgical apparatus comprising:
an elongated body having distal and proximal end portions and a lumen therethrough;
a lens in said lumen;
a transparent tip extending distally from said distal end portion; and
an elongated tube slidable over said elongated body and adapted to cannulate an opening through tissue formed by said tip.

64. The apparatus of claim 63, wherein said elongated tube is flexible.

65. The apparatus of claim 63, wherein said elongated tube is rigid.

66. The apparatus of claim 63, wherein said tip comprises a nipple extending from a distal end of said tip, said nipple adapted to facilitate dissection.

67. The apparatus of claim 63, further comprising a camera proximal of said elongated body, said camera adapted to provide images of light passing through said lens.

68. The apparatus of claim 63, wherein said elongated tube has a length of about two-thirds of a length of said elongated body.

69. A surgical instrument for performing endoscopic functions, said instrument comprising:
an elongated body having distal and proximal end portions and a lumen therethrough;
a lens in said lumen; and
a transparent tip extending distally from said distal end portion, said transparent tip having a distal end portion having a first cross-sectional area larger than a second cross-sectional area of said tip at a location proximal of said distal end portion of said tip, wherein images are viewable through said elongated body, lens and transparent tip.

70. The surgical instrument of claim 69, wherein said distal end portion of said tip is ball-shaped.

71. The surgical instrument of claim 69, wherein said transparent tip comprises a first transparent tip, said instrument further comprising a second transparent tip interchangeable with said first transparent tip, said second transparent tip having a blunt exterior distal surface.

72. The surgical instrument of claim 71, wherein said second transparent tip further comprises a nipple extending from said blunt exterior distal surface, said nipple being adapted to facilitate dissection.

73. Surgical apparatus comprising:
an endoscope having an elongated body and a lumen therethrough, a lens in said lumen, a distal end portion of said endoscope including a distal end portion of said elongated body and a tip extending distally from said elongated body, wherein images are viewable through said tip, lens and lumen; and
an elongated lead cinched over said distal end portion of said endoscope and extending proximally therefrom.

74. The surgical apparatus of claim 73, wherein said elongated lead comprises a snare catheter, said snare catheter comprising an elongated tube, a suture line extending through said elongated tube of said snare catheter and out of both proximal and distal end openings of said tube, and a loop formed at a distal end of said suture line.

75. The surgical apparatus of claim 74, further comprising a lock configured to assume an unlocked configuration in which said suture line and said elongated tube of said snare catheter are freely slidable with respect to one another, and a locked configuration in which said suture line and said elongated tube of said snare catheter are prevented from sliding with respect to one another.

76. The surgical apparatus of claim 75, wherein said lock is integral with a proximal end portion of said snare catheter.

77. The surgical apparatus of claim 73, wherein said tip comprises a transparent tip having a blunt, exterior distal surface.

78. The surgical apparatus of claim 77, wherein said tip further comprises a nipple extending from said blunt, exterior surface, said nipple being adapted to facilitate dissection.

79. The surgical apparatus of claim 73, wherein said tip has a distal end portion having a first cross-sectional area larger than a second cross-sectional area of said tip at a location proximal of said distal end portion of said tip.

80. The surgical apparatus of claim 73, wherein said tip comprises a ball-shaped distal end portion.

81. Surgical apparatus comprising:
a dissecting endoscope having an elongated body having distal and proximal end portions and a lumen therethrough; a lens in said lumen; and a transparent tip extending distally from said distal end portion; and
a tube having an inside diameter larger than an outside diameter of said elongated body, and slidable over said elongated body, said tube being adapted to be mounted on said elongated body prior to dissecting an opening through tissue by said dissecting endoscope, and to be slid distally with respect to said elongated body and through an opening established by said dissecting endoscope, thereby cannulating the opening, even after removal of said dissecting endoscope therefrom.

82. The surgical apparatus of claim 81, wherein said tube is shorter than said elongated body.

83. The surgical apparatus of claim 82, wherein said tube has a length about two-thirds of a length of said elongated body.

84. The surgical apparatus of claim 81, wherein said transparent tip comprises a blunt, exterior distal surface.

85. The surgical apparatus of claim 84, wherein said tip further comprises a nipple extending from said blunt, exterior surface.

86. The surgical apparatus of claim 81, wherein said tube is longer than said elongated body.

87. The surgical apparatus of claim 86, wherein said tube includes at least one slit between proximal and distal ends of said tube, said at least one slit being sufficiently long to form an opening to slidably receive said elongated body.

88. The surgical apparatus of claim 87, wherein said tube is spirally slit along a least a portion of the length of said tube.

89. The surgical apparatus of claim 88, wherein said tube is spirally slit along an entire length of said tube.

90. The surgical apparatus of claim 81, wherein said tube comprises an expandable length tube, said tube being compressable to a length less than a length of said elongated body, said tube being expandable to a length greater than a length of said elongated body.

91. The surgical apparatus of claim 90, wherein said expandable length tube comprises a corrugated tube.

92. The surgical apparatus of claim 90, further comprising a tube having a fixed length and having an inside diameter larger than said outside diameter of said elongated body, and slidable over said elongated body, said fixed length tube having an outside diameter smaller than said inside diameter of said expandable length tube, said expanding length tube being slidable over said fixed length tube and compressable to a compressed length less than a length of said fixed length tube.

93. The surgical apparatus of claim 92, wherein a distal end portion of said fixed length tube comprises a first tube connector and a distal end portion of said expandable length tube comprises a second tube connector, wherein upon sliding said expandable length tube over said fixed length tube and compressing said expandable length tube, said first and second connectors are mateable to form a connection, thereby maintaining said expandable length tube in a compressed configuration.

94. The surgical apparatus of claim 93, wherein said fixed length tube further comprises a stop at a proximal end thereof, said stop configured to prevent said expandable length tube from passing thereover.

95. Surgical apparatus comprising:
an elongated lead having sufficient length to extend from an opening in a right chest of a patient and out of the patient's body at a proximal end of said lead, around four pulmonary veins of the patient and back out of the opening in the right side of the patient at a distal end of said elongated lead, said elongated lead further comprising a first connector at a distal end thereof; and
an elongated instrument having sufficient length to extend through said opening, or a second opening in the right side of the patient to connect with said lead in an oblique pericardial sinus of the patient, said elongated instrument further comprising a second connector, wherein a connection between said elongated instrument and said elongated lead is made via said first and second connectors.

96. The surgical apparatus of claim 95, wherein said first connector comprises a loop extending from said distal end.

97. The surgical apparatus of claim 96, wherein said loop is controllable from a proximal end of said elongated lead to vary a diameter of said loop, and said second connector comprises a tip of said elongated instrument, said loop being cinchable around a distal end portion of said elongated instrument, against a portion of said tip.

98. The surgical apparatus of claim 97, wherein said portion of said tip comprises a shoulder formed at a proximal end of said tip, an outside diameter of said proximal end of said tip being greater than an outside diameter of a portion of said elongated instrument proximally adjacent said proximal end of said tip.

99. The surgical apparatus of claim 97, wherein said portion of said tip comprises a proximal portion of said tip having a first diameter, said tip further comprising a distal portion having a second diameter, wherein said second diameter is greater than said first diameter.

100. The surgical apparatus of claim 99, wherein said distal portion is ball-shaped.

101. The surgical apparatus of claim 95, wherein said elongated instrument comprises an endoscope, said endoscope having a distal tip through which said first connector may be visualized as said endoscope is advanced into the oblique pericardial sinus.

102. The surgical apparatus of claim 96, wherein said elongated instrument comprises an endoscope, said endoscope having a distal tip through which said first connector may be visualized to align said second connector therewith as said endoscope is advanced into the oblique pericardial sinus.

103. The surgical apparatus of claim 99, wherein said elongated instrument comprises an endoscope, and wherein said first connector may be visualized as said second connector is passed therethrough and as said first connector is cinched down on said second connector.

104. The surgical apparatus of claim 95, further comprising an ablation probe adapted to be connected to a proximal end portion of said elongated lead.

105. The surgical apparatus of claim 104, wherein said ablation probe has sufficient length to completely surround the four pulmonary veins.

106. The surgical apparatus of claim 104, wherein said ablation probe is configured to apply microwave energy to form a lesion in tissue.

107. The surgical apparatus of claim 95, wherein said elongated lead comprises a catheter, with a suture line extending through both proximal and distal ends of said catheter, and said first connector comprises a suture loop formed at a distal end of said suture line.

108. The surgical apparatus of claim 107, wherein said elongated instrument comprises an endoscope having a tip through which said suture loop may be visualized as said tip approaches said suture loop in the oblique pericardial sinus, said suture loop being configured to pass over at least a portion of said tip and cinch down to connect said endoscope to said elongated lead.

109. The surgical apparatus of claim 95, wherein said elongated instrument comprises an endoscope and said second connector comprises a tip of said endoscope.

110. The surgical apparatus of claim 109, wherein said tip comprises a dissecting tip, a proximal end of said tip having an outside diameter greater than an outside diameter of a portion of said endoscope joining said proximal end of said tip on a proximal side thereof.

111. The surgical apparatus of claim 109, wherein said tip has a ball-shaped distal portion.

112. The surgical apparatus of claim 95, wherein said first and second connectors comprise rigid loops said rigid loops being interlinkable to form a clasp, thereby connecting said elongated lead with said elongated instrument.

113. The surgical apparatus of claim 112, wherein said elongated instrument comprises an endoscope, said endoscope having a transparent tip from which said rigid loop extends, wherein said endoscope is configured to visualize interlinking of said loops through said tip.

114. The surgical apparatus of claim 95, wherein said elongated lead comprises a tube having at least one slit between proximal and distal ends of said tube, said at least one slit being sufficiently long to form an opening to slidably receive a dissecting endoscope.

115. The surgical apparatus of claim 114, wherein said tube is spirally slit along a least a portion of the length of said tube.

116. The surgical apparatus of claim 115, wherein said tube is spirally slit along an entire length of said tube.

117. The surgical apparatus of claim 114, wherein said first connector comprises a loop extending from a distal end of said tube.

118. The surgical apparatus of claim 117, wherein said loop is controllable proximally of a proximal end of said tube, to vary a diameter of said loop.

119. The surgical apparatus of claim 114, further comprising a catheter running an entire length of said tube, inside of said tube.

120. The surgical apparatus of claim 119, further comprising a suture line running an entire length within said catheter and extending through both proximal and distal ends of said catheter, and said first connector comprises a suture loop formed at a distal end of said suture line.

121. The surgical apparatus of claim 120, wherein said elongated instrument comprises an endoscope.

122. The surgical apparatus of claim 95, wherein said first and second connectors comprise mating threads.

123. The surgical apparatus of claim 122, wherein said elongated instrument comprises an endoscope, said endoscope having a transparent distal tip, wherein said distal tip includes said threads of said second connector.

124. The surgical apparatus of claim 123, wherein said endoscope is configured to view a connection being made between said first and second connectors, through said distal tip.

125. The surgical apparatus of claim 123, wherein said distal tip is a tip configured for use in dissecting tissue.

126. The surgical apparatus of claim 123, wherein said tip is removable for interchanging with a different tip.

127. The surgical apparatus of claim 95, wherein said elongated lead comprises a tube having at least two separate segments, said segments being joined by tube connectors formed on proximal and distal portions of said segments, respectively.

128. The surgical apparatus of claim 95, wherein said elongated lead comprises an expandable length tube, said tube being compressable to a compressed length, said expandable length tube having an expanded length when not compressed, said expanded length being greater that said compressed length.

129. The surgical apparatus of claim 128, wherein said expandable length tube comprises a corrugated tube.

130. The surgical apparatus of claim 128, further comprising a tube having a fixed length and having an outside diameter smaller than an inside diameter of said expandable length tube, said expandable length tube being slidable over said fixed length tube and compressable to said compressed length, wherein said compressed length is less than a length of said fixed length tube.

131. The surgical apparatus of claim 130, wherein a distal end portion of said fixed length tube comprises a first tube connector and a distal end portion of said expandable length tube comprises a second tube connector, wherein upon sliding said expandable length tube over said fixed length tube and compressing said expandable length tube, said first and second connectors are mateable to form a connection, thereby maintaining said expandable length tube in a compressed configuration.

132. The surgical apparatus of claim 131, wherein said fixed length tube further comprises a stop at a proximal end thereof, said stop configured to prevent said expandable length tube from passing thereover.

133. A surgical device comprising:
an elongated tube having sufficient length to extend from an opening in a right chest of a patient and out of the patient's body at a proximal end of said tube, around four pulmonary veins of the patient and back out of the opening in the right side of the patient at a distal end of said tube, said tube further comprising at least one slit between proximal and distal ends of said tube, said at least one slit being sufficiently long to form an opening to slidably receive an endoscope.

134. The surgical device of claim 133, wherein said tube is spirally slit along a least a portion of the length of said tube.

135. The surgical device of claim 134, wherein said tube is spirally slit along an entire length of said tube.

136. The surgical device of claim 133, wherein said device further comprises a first connector at a distal end of said tube, said first connector adapted to be joined with a second connector while positioned in an oblique pericardial sinus of a patient.

137. The surgical device of claim 136, wherein said first connector comprises a loop extending from a distal end of said tube.

138. The surgical device of claim 137, wherein said loop is controllable proximally of a proximal end of said tube, to vary a diameter of said loop.

139. The surgical device of claim 133, further comprising a catheter running an entire length of said tube, inside of said tube.

140. The surgical device of claim 139, further comprising a suture line running an entire length within said catheter and extending through both proximal and distal ends of said catheter, and said first connector comprises a suture loop formed at a distal end of said suture line.

141. The surgical device of claim 133, wherein said elongated tube has an inside diameter sufficiently large to pass an ablation probe therethrough.

142. Surgical apparatus comprising:
an elongated lead having sufficient length to extend from an opening in a right chest of a patient and out of the patient's body at a proximal end of said lead, around four pulmonary veins of the patient and back out of the opening in the right side of the patient at a distal end of said elongated lead, said elongated lead comprising a first tube and a second tube, a proximal end of said first tube being connectable with a distal end of said second tube; and
an elongated instrument having sufficient length to extend through said opening and into a transverse pericardial sinus of the patient, wherein said first tube is configured to be freely slidable over said elongated instrument, and said first tube has a length less than a length of said elongated instrument, so that a distal end of said elongated instrument extends beyond a distal end of said first tube when said first tube is slid over said elongated instrument.

143. The surgical apparatus of claim 142, further comprising a first connector at a distal end of said first tube, said first connector adapted to make a connection with an elongated instrument inserted into an oblique pericardial sinus of the patient when said distal end portion of said first tube has been advanced into the oblique pericardial sinus.

144. The surgical apparatus of claim 143, wherein the elongated instrument inserted into the oblique pericardial sinus comprises said elongated instrument having sufficient length to extend through said opening and into a transverse pericardial sinus of the patient, said instrument comprising a distal end portion having a second connector, said second connector being mateable with said first connector to form said connection.

145. The surgical apparatus of claim 143, wherein said elongated instrument comprises a dissecting endoscope, and wherein said dissecting endoscope is configured to dissect through a pericardium of the patient while said first tube is mounted over said dissecting endoscope, prior to advancing said first tube through the pericardium and into the transverse pericardial sinus.

146. The surgical apparatus of claim 144, wherein said elongated instrument comprises an endoscope, said endoscope having a distal tip comprising said second connector.

147. The surgical apparatus of claim 146, wherein said first and second connectors comprise a set of mating threads.

148. Surgical apparatus comprising:
an expandable length tube having an expanded length configuration having sufficient length to extend from an opening in a right chest of a patient through a transverse pericardial sinus of the patient, around left pulmonary veins of the patient and into an oblique pericardial sinus of the patient, and a compressed length configuration having a length shorter than said expandable length configuration, said tube having an opening therethrough configured to pass an ablation probe therethrough.

149. The surgical apparatus of claim 148, further comprising a lumen running an entire length of said tube, inside of said tube, even when said tube is in said expandable length configuration.

150. The surgical apparatus of claim 149, further comprising a suture line running an entire length within said lumen and extending through both proximal and distal ends of said lumen, and a suture loop formed at a distal end of said suture line.

151. The surgical apparatus of claim 148 wherein said tube comprises a corrugated tube.

152. The surgical apparatus of claim 151, further comprising a catheter extending through slip fit openings in corrugations of said corrugated tube, outside a minor diameter of said corrugated tube, wherein said catheter spans an entire length of said tube, even when said tube is in said expandable length configuration.

153. The surgical apparatus of claim 152, further comprising a suture line running an entire length within said catheter and extending through both proximal and distal ends of said catheter, and a suture loop formed at a distal end of said suture line.

154. The surgical apparatus of claim 151, further comprising a lumen running an entire length of said corrugated tube, inside of said corrugated tube, even when said corrugated tube is in said expandable length configuration.

155. The surgical apparatus of claim 154, further comprising a suture line running an entire length within said lumen and extending through both proximal and distal ends of said lumen, and a suture loop formed at a distal end of said suture line.

156. The surgical apparatus of claim 148, further comprising a tube having a fixed length and having an outside diameter smaller than an inside diameter of said expandable length tube, said expandable length tube being slidable over said fixed length tube and compressable to said compressed length, wherein said compressed length is less than a length of said fixed length tube.

157. The surgical apparatus of claim 156 wherein a distal end portion of said fixed length tube comprises a first tube connector and a distal end portion of said expandable length tube comprises a second tube connector, wherein upon sliding said expandable length tube over said fixed length tube and compressing said expandable length tube, said first and second connectors are mateable to form a connection, thereby maintaining said expandable length tube in a compressed configuration.

158. The surgical apparatus of claim 156, wherein said fixed length tube further comprises a stop at a proximal end thereof, said stop configured to prevent said expandable length tube from passing thereover.

159. The surgical apparatus of claim 148, further comprising an elongated instrument having sufficient length to extend through said opening and into the transverse pericardial sinus while a proximal portion of said instrument remains outside the body of the patient; said expandable length tube being slidable over the distal end of said instrument to be slidably mounted on said elongated instrument, wherein when said expandable length tube is mounted on said elongated instrument and compressed to said compressed length configuration, said distal end of said elongated instrument extends beyond a distal end of said expandable length tube in said compressed length configuration.

160. The surgical apparatus of claim 159, wherein said distal end of said elongated instrument comprises a dissecting tip, said elongated instrument being configured to dissect an opening through the pericardium, leading to the transverse pericardial sinus, prior to inserting a distal end portion of said elongated instrument into the transverse pericardial sinus and prior to inserting a distal end of said expandable length tube into the transverse pericardial sinus.

161. The surgical apparatus of claim 148, wherein said elongated instrument comprises an endoscope and said distal end of said elongated instrument comprises a transparent tip thorough which procedures carried out with said elongated instrument may be viewed from outside of the body.

162. The surgical apparatus of claim 160, wherein said elongated instrument comprises a dissecting endoscope and said dissecting tip is transparent to permit said dissecting and inserting to be viewed therethrough from outside of the body.

163. The surgical apparatus of claim 156, further comprising an elongated instrument having sufficient length to extend through said opening and into the transverse pericardial sinus while a proximal portion of said instrument remains outside the body of the patient; said fixed length tube being slidable over the distal end of said instrument to be slidably mounted on said elongated instrument while said elongatable length tube is mounted on said fixed length tube in a compressed configuration, wherein when said expandable length tube in said compressed length configuration and said fixed length tube are mounted on said elongated instrument, said distal end of said elongated instrument extends beyond a distal end of said expandable length tube in said compressed length configuration.

164. The surgical apparatus of claim 163, wherein said distal end of said elongated instrument comprises a dissecting tip, said elongated instrument being configured to dissect an opening through the pericardium, leading to the transverse pericardial sinus, prior to inserting a distal end portion of said elongated instrument into the transverse pericardial sinus and prior to inserting a distal end of said expandable length tube into the transverse pericardial sinus.

165. The surgical apparatus of claim 163, wherein said elongated instrument comprises an endoscope and said distal end of said elongated instrument comprises a transparent tip thorough which procedures carried out with said elongated instrument may be viewed from outside of the body.

166. The surgical apparatus of claim 164, wherein said elongated instrument comprises a dissecting endoscope and said dissecting tip is transparent to permit said dissecting and inserting to be viewed therethrough from outside of the body.

167. A surgical device comprising:
an elongated tubular instrument having a main tube with sufficient length to extend through an opening in the chest wall of a patient, through the pericardium of the patient and into the transverse pericardial sinus while a proximal portion of said instrument remains outside the body of the patient;
a transparent distal tip mounted to a distal end of said main tube; and
a second tube running parallel to said main tube and having an inside diameter sufficient to pass a snare catheter therethrough.

168. The surgical device of claim 167, wherein said instrument comprises an endoscope, a proximal portion of said instrument comprising a camera, wherein images are generated from light passing through said transparent distal tip.

169. The surgical device of claim 167, wherein said distal tip is configured to dissect tissue.

170. The surgical device of claim 167, wherein said distal tip comprises an enlarged ball shaped distal portion.

171. The surgical device of claim 167, further comprising a snare catheter extending through said second tube and a suture loop formed at a distal end of said snare catheter.

172. The surgical device of claim 169, wherein said distal end of said distal tip comprises a nipple extending from a distal surface of said distal tip.

173. A surgical device comprising:
an elongated ablation probe having a proximal end portion and a distal end portion; and
an elongated, flexible tube having a proximal end portion and a distal end portion, said proximal end portion of said tube being releasably connectable to said distal end portion of said probe.

174. The surgical device of claim 173, wherein said flexible tube comprises a torque tube, said torque tube being flexible in bending about a longitudinal axis of said torque tube, but rigid with respect to torsion about said longitudinal axis.

175. The surgical device of claim 173, further comprising an opening formed in said proximal end portion of said flexible tube, said opening sized to receive an instrument therethrough, said tube further comprising an opening in a distal end portion thereof.

176. The surgical device of claim 175, wherein a lumen connecting said opening in said proximal end portion and said opening in said distal end portion comprises a reduced cross-sectional area in at least one location proximal of said opening in said distal end portion, said reduced cross-sectional area being less than an area of said opening in said proximal end portion.

177. The surgical device of claim 175, wherein a lumen connecting said opening in said proximal end portion and said opening in said distal end portion comprises a reduced cross-sectional area in at least one location proximal of said opening in said distal end portion to prevent passage of an instrument therethrough, wherein said instrument is insertable through said opening in said proximal end portion and said lumen, until contacting said reduced cross-sectional area.

178. The surgical device of claim 173, wherein said distal end portion of said tube comprises a connector configured to connect with a connector on an instrument inserted into an oblique pericardial sinus of the patient.

179. The surgical device of claim 173, further comprising a suture loop extending from said distal end portion of said tube.

180. The surgical device of claim 175, further comprising a catheter inserted through said opening in said proximal end portion of said tube, a distal end portion of said catheter extending from said opening in said distal end portion, and a proximal end portion of said catheter extending proximally from said opening in said proximal end portion of said tube, and wherein said catheter comprises a connector at a distal end portion thereof.

181. The surgical device of claim 180, wherein said catheter comprises a snare catheter, said snare catheter comprising a suture line extending therethrough, said suture line comprising a loop at a distal end thereof.

182. A surgical device comprising:
an elongated tubular instrument having a main tube with sufficient length to extend through an opening in the chest wall of a patient, through the pericardium of the patient and into the transverse pericardial sinus while a proximal portion of said instrument remains outside the body of the patient;
a transparent distal tip mounted to a distal end of said main tube;
a lumen extending through said main tube and opening to a proximal end of said device, said lumen configured to receive an endoscope to permit viewing by said endoscope through said transparent distal tip; and
a second lumen extending through said main tube and opening at a proximal end portion of said device to receive a snare catheter therethrough.

183. The surgical device of claim 182, wherein said tip includes an opening therethrough, said opening connecting with said second lumen, wherein a distal end of said snare catheter is passable through said opening.

184. The surgical device of claim 182, further comprising a third lumen extending through said main tube and connecting an opening though said distal tip with a suction luer opening at a proximal end portion of said device.

185. The surgical device of claim 182, further comprising an endoscope positioned in said lumen configured to receive an endoscope.

186. The surgical device of claim 182, further comprising a snare catheter positioned in said second lumen and a snare extending distally of said main tube.

187. The surgical device of claim 185, further comprising a snare catheter positioned in said second lumen and a snare extending distally of said main tube.

188. The surgical device of claim 183, further comprising a snare catheter positioned in said second lumen and extending through said opening.

189. The surgical device of claim 182, wherein said distal tip comprises an enlarged ball shaped distal portion.

190. The surgical device of claim 182, wherein said distal end of said distal tip comprises a nipple extending from a distal surface of said distal tip.

191. The surgical device of claim 182, further comprising an elongated insert configured to be positioned within said main tube, said insert comprising a groove extending over the length of said insert, said grooves, together with an inside wall of said main tube forming said lumen and said second lumen.
